(19) United States
(12) Patent Application Publication
(10) Pub. No.: US 2025/0367249 A1
(43) Pub. Date: Dec. 4, 2025

(54) HOMOGENEOUS EXTRACT-INFUSED CANNABIS PRODUCTS AND METHODS FOR PREPARATION THEREOF

(71) Applicant: ANC INC., Edmonton (CA)

(72) Inventors: Tairance Rutter, Edmonton (CA); Thomas Facciolo, Edmonton (CA); James Clarke, Edmonton (CA); Clayton Bordeniuk, Edmonton (CA); Raquel Razzera Huerta, Saint Albert (CA)

(73) Assignee: ANC INC., Edmonton, AB (CA)

(21) Appl. No.: 18/876,133

(22) PCT Filed: Jun. 16, 2023
(86) PCT No.: PCT/CA2023/050841

§ 371 Date: Dec. 17, 2024

Related U.S. Application Data
(60) Provisional application No. 63/353,422, filed on Jun. 17, 2022.

(51) Int. Cl. A61K 36/185

(52) U.S. Cl. CPC A61K 36/3482

(57) ABSTRACT

Homogeneous extract-infused cannabis biomasses, methods for their preparation, and products produced therefrom, such as pre-rolled joints, are provided. An exemplary method comprises steps of providing a cannabis extract; heating the cannabis extract; providing a milled cannabis plant material; applying the heated cannabis extract to the milled cannabis plant material using a predefined ratio of the cannabis plant material to the heated cannabis extract to provide an extract-applied plant material; and mixing the extract-applied plant material to provide a homogeneous extract-infused cannabis biomass, wherein there is less than a 15% variability in a target concentration of the at least one biomass cannabinoid, the at least one biomass terpene, or any combination thereof throughout a prepared batch and/or between alternate batches of the homogeneous extract-infused cannabis biomass.

### CROSS-REFERENCE TO RELATED APPLICATION

This application claims priority to and benefit from U.S. Patent Application Ser. No. 63/353,422 filed on Jun. 17, 2022, which is hereby incorporated by reference in its entirety.

### TECHNICAL FIELD

The present disclosure relates generally to homogeneous extract-infused cannabis products. More specifically, the present disclosure relates to products comprising a homogeneous extract-infused cannabis biomass, and methods for preparation thereof.

### BACKGROUND

Owing to the physiological outcomes associated with consuming cannabis products, due to the secondary metabolites present therein, cannabis has been widely used as a recreation drug. As laws are relaxed across various countries and jurisdictions for cannabis use and/or cannabis possession, there now exists the opportunity to explore the potential to optimize the use of cannabis and cannabis products. One of the most common and desirable methods of consuming cannabis is by smoking. Smoking cannabis plant material involves burning the flower and inhaling the bioactive components of the plant that are released. However, the availability of bioactive components is limited by plant strain.

The emergence of cannabis extracts, which contain a higher concentration of certain secondary metabolites when compared to the cannabis plant material from which they were derived, comprises products such as, for example, cannabis distillates, cannabis concentrates, cannabis oils, cannabis isolates, and cannabis resins. Cannabis extracts can be optimized to contain specific secondary metabolite profiles, including cannabinoids and terpenes, which are associated with the physiological outcomes of cannabis use. The optimized secondary metabolite profiles of cannabis extracts allow for the production of products with accurate and reproducible metabolite profiles, which may improve the user experience by altering, for example, the physiological outcome or flavour of the product when consumed. To this end, cannabis extracts are often consumed by methods such as vaping, which may not be a desirable consumption method. Moreover, a user may smoke an increased amount of a smokable product, such as, for example, a joint, in order consume the desired amount of secondary metabolites to elicit the desired outcome. As such, the user may consume more smoke, which may have a detrimental impact on the long-term health of the user, to achieve the desired outcome. To this end, there is a need for a smokable cannabis product, that does not comprise vaping, that produces a desired effect with consumption of less product. There is further a need that such product be reproducible in its composition, e.g. within batches, between batches and within each individual product.

The combination of cannabis extracts and cannabis plant material may provide a smokable product with a higher amount or a target amount of specific secondary metabolites, such as cannabinoids and/or terpenes. However, consistent and reproducible manufacture of such products, including accuracy and consistency of dosing of secondary metabolites, has been problematic both between batches and within batches or individual products. Additionally, the use of liquid cannabis extracts, such as a cannabis distillate, may have unique challenges when compared to the use of solid cannabis extracts, such as hash, kief, or wax, to provide extract-infused cannabis products. Providing a homogeneous extract-infused product from a liquid cannabis concentrate, such as cannabis distillate or a cannabis concentrate, has thus far proved elusive.

Current techniques to provide a liquid extract-infused cannabis plant material do not provide a homogeneous extract-infused product. For example, the injection of liquid cannabis extract provides liquid to a specific portion (e.g. the central portion) of the product, such as a joint, whereas other portions do not receive or contain an equivalent amount or concentration of the cannabis extract. Unequal distribution of the cannabis extract may have several effects on the product, including undesirable consumption, such as uneven burning and/or clogging (e.g. due to impaired burning), undesired physiological outcomes, uneven delivery of active payload (e.g. cannabinoid) to the user, decreased product stability, and/or an inability to adhere to local cannabis related regulations.

To this end, extract-infused cannabis products, such as pre-rolled joints, that provide a homogeneous cannabinoid and/or terpene concentration both within an individual pre-rolled joint, across a single batch, and across multiple batches are required, as well as efficient methods for their production.

### SUMMARY

Described herein are extract-infused cannabis products, such as pre-rolled joints, that include a homogeneous extract-infused cannabis biomass having low variability in a target concentration of at least one cannabinoid and/or at least one terpene throughout a single product and/or between batches, as well as methods for the preparation thereof.

Government regulations in, for example, Canada require the measured cannabinoid concentration of a cannabis extract, such as a homogeneous extract-infused cannabis biomass or product to fall within 15% of the cannabinoid amount stated on the packaging. As such, methods to ensure the accuracy and reproducibility in the preparation of extract-infused cannabis products may provide a reliable means to produce an extract-infused product that falls within the 15% variability required for sale, and ensure that the user consistently achieves the desired outcome following use of the extract-infused cannabis product. As well, the ability to consistently produce and provide a homogeneous extract-infused cannabis product with less than 15% variability may provide a cost savings to producers, as smaller variability ranges allow the shipment of exact quantities for production, which may minimize waste and labour. Additionally, to improve the quality of the product and the user experience, homogeneous extract-infused cannabis biomass or products, such as pre-rolled joints, which provide a homogeneous amount of at least one cannabinoid and/or at least one terpene throughout an individual product, across a batch of the product or across multiple batches of the product are required.

To this end, preparing homogeneous extract-infused cannabis products accurately and reproducibly with respect to the cannabinoid concentration and/or the terpene concentration has proved challenging, which impacts the ability to prepare extract-infused cannabis products, as well as distribute and sell such products owing to local regulations and the desired experience by users.

Alternative, additional and/or improved extract-infused cannabis products and biomasses, as well as methods for their preparation, are desirable.

Herein we describe homogeneous extract-infused cannabis products, as well as methods for preparation thereof, which may provide an accurate and reproducible cannabinoid concentration and/or terpene concentration in the homogeneous extract-infused cannabis biomass or product.

In certain embodiments herein, there are provided methods for preparing a homogeneous extract-infused cannabis biomass, comprising providing a cannabis extract having a predefined concentration of at least one extract-derived cannabinoid, at least one extract-derived terpene, or any combination thereof; heating the cannabis extract at a temperature of up to 120° C. to provide a heated cannabis extract, wherein the heated cannabis extract is in a substantially liquid or flowable state; providing a cannabis plant material that having a predefined concentration of at least one plant-derived cannabinoid, at least one plant-derived terpene, or any combination thereof, wherein the cannabis plant material is a milled or comminuted cannabis plant material; applying the heated cannabis extract to the cannabis plant material using a predefined ratio of the cannabis plant material to the heated cannabis extract to provide an extract-applied plant material, wherein the predefined ratio provides a target amount of at least one biomass cannabinoid, at least one biomass terpene, or any combination thereof within the extract-applied plant material; and mixing the extract-applied plant material to provide a homogeneous extract-infused cannabis biomass, wherein there is less than a 15% variability in a target concentration of the at least one biomass cannabinoid, the at least one biomass terpene, or any combination thereof throughout a prepared batch and/or between alternate batches of the homogeneous extract-infused cannabis biomass.

In certain embodiments, there may be less than a 10% variability in the target concentration of the at least one biomass cannabinoid, the at least one biomass terpene, or any combination thereof throughout the prepared batch, between the alternate batches, or both. In certain embodiments, there may be less than a 7.5% variability in the target concentration of the at least one biomass cannabinoid, the at least one biomass terpene, or any combination thereof throughout the prepared batch, between the alternate batches, or both. In certain embodiments, there may be less than a 5% variability in the target concentration of the at least one biomass cannabinoid, the at least one biomass terpene, or any combination thereof throughout the prepared batch, between the alternate batches, or both. In certain embodiments, there may be less than a 4% variability in the target concentration of the at least one biomass cannabinoid, the at least one biomass terpene, or any combination thereof throughout the prepared batch, between the alternate batches, or both.

In certain embodiments, there may be between about 0.5% to about 10% variability in the target concentration of the at least one biomass cannabinoid, the at least one biomass terpene, or any combination thereof throughout the prepared batch, between the alternate batches, or both. In certain embodiments, there may be between about 3% to about 7% variability in the target concentration of the at least one biomass cannabinoid, the at least one biomass terpene, or any combination thereof throughout the prepared batch, between the alternate batches, or both. In certain embodiments, the homogeneous extract-infused cannabis biomass may have substantially no variability in the target concentration of the at least one biomass cannabinoid, the at least one biomass terpene, or any combination thereof throughout the prepared batch, between the alternate batches, or both.

In certain embodiments, the target amount may be of the at least one biomass cannabinoid, the at least one biomass terpene, or both, and the target amount of the at least one biomass cannabinoid may be obtained by the at least one extract-derived cannabinoid, the at least one plant-derived cannabinoid, or both; and the target amount of the at least one biomass terpene may be obtained by the at least one extract-derived terpene, the at least one plant-derived terpene, or both.

In certain embodiments, the target amount may be of the at least one biomass cannabinoid, and the target amount may be obtained by the at least one extract-derived cannabinoid or the at least one plant-derived cannabinoid.

In certain embodiments, the target amount may be of the at least one biomass cannabinoid, and the target amount may be obtained by a combination of the at least one extract-derived cannabinoid and the at least one plant-derived cannabinoid.

In certain embodiments, the target amount may be of the at least one biomass terpene, and the target amount may be obtained by the at least one extract-derived terpene or the at least one plant-derived terpene.

In certain embodiments, the target amount may be of the at least one biomass cannabinoid and the at least one biomass terpene, and the target amount may be obtained by a combination of (i) the at least one extract-derived cannabinoid and the at least one extract-derived terpene; and (ii) the at least one plant-derived cannabinoid and the at least one plant-derived terpene.

In certain embodiments, the method may further comprise a step of providing at least one additive to the cannabis extract prior to the applying of the heated cannabis extract to the cannabis plant material. In certain embodiments, the at least one additive may comprise at least one additive terpene, at least one additive cannabinoid, or any combination thereof, wherein the at least one additive may be added to the cannabis extract at a predefined amount.

In certain embodiments, which comprise providing the at least one additive terpene, the at least one additive terpene may be provided at a weight ratio of the at least one additive terpene to the cannabis extract of no greater than 0.3, and preferably the weight ratio may be between about 0.15 and about 0.25.

In certain embodiments, the target amount may be of the at least one biomass cannabinoid, the at least one biomass terpene, or both, and the target amount of the at least one biomass cannabinoid may be obtained from one or more of the at least one extract-derived cannabinoid, the at least one plant-derived cannabinoid, and the at least one additive-derived cannabinoid; and the target amount of the at least one biomass terpene may be obtained from one or more of the at least one extract-derived terpene, the at least one plant-derived terpene, and the at least one additive-derived terpene.

In certain embodiments, the target amount may be of the at least one biomass cannabinoid, and the target amount may be obtained by a combination of the at least one additive-derived cannabinoid and the at least one extract-derived cannabinoid or the at least one plant-derived cannabinoid.

In certain embodiments, the target amount may be of the at least one biomass cannabinoid, and the target amount may be obtained by a combination of the at least one extract-derived cannabinoid, the at least one plant-derived cannabinoid, and the at least one additive-derived cannabinoid.

In certain embodiments, the target amount may be of the at least one biomass terpene, and the target amount may be obtained by a combination of the at least one additive-derived terpene and the at least one extract-derived terpene or the at least one plant-derived terpene.

In certain embodiments, the target amount may be of the at least one biomass terpene, and the target amount may be obtained by a combination of the at least one extract-derived terpene, the at least one plant-derived terpene, and the at least one additive-derived terpene.

In certain embodiments, the target amount may be of the at least one biomass cannabinoid and the at least biomass terpene, and the target amount may be obtained by a combination of (i) the at least one additive-derived cannabinoid and the at least one extract-derived cannabinoid or the at least one plant-derived cannabinoid, and (ii) the at least one additive-derived terpene and the at least one extract-derived terpene or the at least one plant-derived terpene.

In certain embodiments, the target amount may be of the at least one biomass cannabinoid and the at least one biomass terpene, and the target amount may be obtained by a combination of (i) the at least one extract-derived cannabinoid, the at least one plant-derived cannabinoid, and the at least one additive-derived cannabinoid, and (ii) the at least one extract-derived terpene, the at least one plant-derived terpene, and the at least one additive-derived terpene.

In certain embodiments, the mixing may be repeated one or more times to obtain the homogeneous extract-infused cannabis biomass.

In certain embodiments, the step of mixing the extract-applied plant material may comprise a step of freezing or cooling the extract-applied plant material.

In certain embodiments, the method may comprise freezing the extract-applied plant material at a temperature between about −30° C. to about −1° C. to produce a frozen extract-applied plant material.

In certain embodiments, the method may comprise mixing the frozen extract-applied plant material to provide the homogeneous extract-infused cannabis biomass.

In certain embodiments, the method may further comprise, after the mixing, a step of screening the homogeneous extract-infused cannabis biomass to improve homogeneity.

In certain embodiments, the applying may be by pouring, sprinkling or showering the heated cannabis extract onto the cannabis plant material.

In certain embodiments, the mixing may be by rotational mixing, blending, stirring, beating, kneading, or any combination thereof of the extract-applied plant material.

In certain embodiments, the applying and the mixing steps may be performed simultaneously.

In certain embodiments, the mixing may be performed in a mixer comprising a flat beater attachment and the flat beater attachment mixes the cannabis plant material and the heated cannabis extract. In certain embodiments, the flat beater attachment mixes the cannabis plant material and the heated cannabis extract at between about 80 rpm and about 120 rpm.

In certain embodiments, in the step of heating the cannabis extract, the temperature may be between about 60° C. and about 120° C. In certain embodiments, in the step of heating the cannabis extract, the temperature may be between about 60° C. and about 100° C. In certain embodiments, in the step of heating the cannabis extract, the temperature may be about 80° C.

In certain embodiments, the cannabis extract may contain minimal or no residual solvent used in an extraction process.

In certain embodiments, the cannabis extract may be a cannabis concentrate, a cannabis distillate, a cannabis oil, a cannabis isolate, a cannabis resin, or any combination thereof. In certain further embodiments, the cannabis extract may be a cannabis distillate. In certain embodiments, the cannabis extract may be a cannabis concentrate.

In certain embodiments, the target amount of the biomass cannabinoid may be of tetrahydrocannabiorcol (THCC), Δ9-tetrahydrocannabinol (Δ9-THC), Δ8-tetrahydrocannabinol (Δ8-THC), cannabidiol (CBD), cannabigerol (CBG), cannabichromene (CBC), cannabinol (CBN), cannabicyclol (CBL), cannabielsoin (CBE), cannabivarin (CBV), cannabifuran (CBF), cannabitran (CBT), cannabiripsol (CBR), any acidic forms thereof, or any combination thereof. In certain further embodiments, the target amount of the biomass cannabinoid may be of Δ9-THC, CBD, CBG, any neutral versions thereof, or any combination thereof.

In certain embodiments, the target amount of the biomass terpene may be of bisabolol, borneol, camphene, carene, caryophyllene, eucalyptol, farnesene, geraniol, humulene, limonene, linalool, myrcene, nerolido, farnesene, pinene, terpineol, valencene, hemiterpenoids, monoterpenoid, sesquiterpenoid, diterpenoid, trans-caryophyllene, sesterterpenoid, triterpenoid, tetraterpenoid, polyterpenoid, citral, menthol, camphor, salvinorin A, ginkgolide or any combination thereof. In certain embodiments, the target amount of the biomass terpene may be of caryophyllene, farnesene, linalool, or any combination thereof.

In certain embodiments, the target amount may be of the at least one biomass terpene, and the target amount may be obtained by a combination of the at least one extract-derived terpene and the at least one plant-derived terpene.

In certain embodiments, the cannabis plant material may be whole plant, buds, flower, flower portions, bark, rhizomes, fruit, or any combination thereof. In certain embodiments, the cannabis plant material may be dried flower.

In certain embodiments, the milled or comminuted cannabis plant material may be of a particle size of less than or equal to 5.0 mm. In certain embodiments, the particle size may be between about 100 μm and about 3.5 mm.

In certain embodiments, a mass of the cannabis extract; the predefined concentration of the at least one extract-derived cannabinoid; the predefined concentration of the at least one extract-derived terpene; a mass of the cannabis plant material; the predefined concentration of the at least one plant-derived cannabinoid; the predefined concentration of the at least one plant-derived terpene; or any combination thereof may be calculated using:

$$E = \frac{[ A \times B ] + [ C \times D ]}{[ A + C ]}$$

wherein A is the mass of the cannabis plant material, B is the concentration of the at least one plant-derived cannabinoid or the concentration of the at least one cannabis plant-derived terpene, C is the mass of the cannabis extract, D is the concentration of the at least one extract-derived cannabinoid or the concentration of the at least one extract-derived terpene, and E is the target concentration of the at least one biomass cannabinoid or the at least one biomass terpene. In certain embodiments, the predefined concentration may be determined in each instance. In certain embodiments, the predefined concentration may be a known quantity, such as, for example, a quantity based on known characteristics of the extract.

In certain embodiments, a length of time for the heating of the cannabis extract may be calculated using:

The length of time for heating of the cannabis extract

$$( s ) = \frac{m \times cp \times Δ T}{P}$$

wherein m is a mass of the cannabis extract, cp is a specific heat capacity of the cannabis extract in J/kg° C., ΔT is a temperature change (° C.) of the cannabis extract; and P is a rate of energy (W) transfer to the cannabis extract.

In certain embodiments, the homogeneous extract-infused cannabis biomass may comprise at least 55% [w/w], at least 60% [w/w], at least 70% [w/w], or at least 75% [w/w], of the cannabis plant material.

In certain embodiments, the variability in the target concentration of the at least one biomass cannabinoid, the at least one biomass terpene, or any combination thereof may be throughout the prepared batch.

In certain embodiments, the variability in the target concentration of the at least one biomass cannabinoid, the at least one biomass terpene, or any combination thereof may be between alternate batches.

In certain embodiments, the variability in the target concentration of the at least one biomass cannabinoid, the at least one biomass terpene, or any combination thereof may be throughout the prepared batch and between alternate batches.

In certain embodiments, the homogeneous extract-infused cannabis biomass may be suitable for use in a pre-rolled joint.

In certain embodiments, the method may comprise a step of providing the homogeneous extract-infused cannabis biomass to a filling device, wherein the filling device may be configured to provide the pre-rolled joint.

In certain embodiments, homogeneous extract-infused cannabis products may be prepared from a homogeneous extract-infused biomass prepared according to a method herein. In certain embodiments, the homogeneous extract-infused cannabis product may be a pre-rolled joint.

In certain embodiments, the homogeneous extract-infused cannabis biomass may be frozen or cooled in order to produce the pre-rolled joint. In certain embodiments, the homogeneous extract-infused cannabis biomass may be frozen or cooled at between about −30° C. to about −10° C.

In certain embodiments, there is provided a homogeneous extract-infused cannabis biomass produced according to a method herein. In certain embodiments, the homogeneous extract-infused cannabis biomass may be for use in a pre-rolled joint.

In certain embodiments, the homogeneous extract-infused cannabis biomass may be provided to a filling device, wherein the filling device may be configured to provide the pre-rolled joint.

In certain embodiments, the extract-infused cannabis biomass may be frozen or cooled in order to produce the pre-rolled joint. In certain embodiments, the extract-infused cannabis biomass may be frozen or cooled at between about −30° C. to about −10° C.

In certain embodiments, there is provided a pre-rolled joint comprising a wrap or paper; a homogeneous extract-infused cannabis biomass contained within the wrap or paper, the homogeneous extract-infused cannabis biomass comprising a target concentration of at least one cannabinoid, at least one terpene, or any combination thereof; and a filter, crutch, tip or mouthpiece disposed at a smoking end of the pre-rolled joint, wherein at least a portion of the target concentration of the at least one cannabinoid, at least one terpene, or combination thereof is from an exogenous source to an originating biomass of the extract-infused cannabis biomass, and wherein there is less than 15% variability in the target concentration of the at least one cannabinoid, at least one terpene, or combination thereof throughout the homogeneous extract-infused cannabis biomass contained within the pre-rolled joint.

In certain embodiments, there may be less than 10% variability in the target concentration of the at least one cannabinoid, at least one terpene, or combination thereof throughout the homogeneous extract-infused cannabis biomass contained within the pre-rolled joint. In certain embodiments, there may be less than 7.5% variability in the target concentration of the at least one cannabinoid, at least one terpene, or combination thereof throughout the homogeneous extract-infused cannabis biomass contained within the pre-rolled joint. In certain embodiments, there may be less than 5% variability in the target concentration of the at least one cannabinoid, at least one terpene, or combination thereof throughout the homogeneous extract-infused cannabis biomass contained within the pre-rolled joint. In certain embodiments, there may be less than 4% variability in the target concentration of the at least one cannabinoid, at least one terpene, or combination thereof throughout the homogeneous extract-infused cannabis biomass contained within the pre-rolled joint.

In certain embodiments, there may be between about 1% to about 10% variability in the target concentration of the at least one cannabinoid, at least one terpene, or combination thereof throughout the homogeneous extract-infused cannabis biomass contained within the pre-rolled joint. In certain embodiments, there may be between about 3% to about 7% variability in the target concentration of the at least one cannabinoid, at least one terpene, or combination thereof throughout the homogeneous extract-infused cannabis biomass contained within the pre-rolled joint. In certain embodiments, the homogeneous extract-infused cannabis biomass may have substantially no variability in concentration of the at least one cannabinoid, at least one terpene, or combination thereof throughout the homogeneous extract-infused cannabis biomass contained within the pre-rolled joint.

In certain embodiments, the homogeneous extract-infused cannabis biomass is as defined herein.

In certain embodiments, the homogeneous extract-infused cannabis is prepared according to a method defined herein.

In certain embodiments, the pre-rolled joint may be free of any residual solvent. In certain embodiments, the pre-rolled joint may be free of any Class 3 solvent. In certain embodiments, the pre-rolled joint may be free of ethanol.

In certain embodiments, the pre-rolled joint may exhibit a smooth even burn.

In certain embodiments, during smoking the pre-rolled joint, the inhaled concentration or potency of any one or more cannabinoids or terpenes may exhibit less than 10% variability from start to finish, may exhibit less than 7.5% variability from start to finish, may exhibit less than 5% variability from start to finish, may exhibit less than 4% variability from start to finish, may exhibit between about 1% and about 10% variability from start to finish, or may exhibit between about 3% and about 7% variability from start to finish.

In certain embodiments, the pre-rolled joint may comprise an additive. In certain embodiments, the additive may comprise at least one additive terpene, at least one additive cannabinoid, or any combination thereof, wherein the additive is provided at a predefined additive amount to provide the target concentration of the at least one cannabinoid, at least one terpene, or combination thereof.

In certain embodiments, the pre-rolled joint may be free of oily residue leaching through to the external surface of the wrap or paper. In certain embodiments, wherein if the pre-rolled joint is taken apart, the biomass of the extract-infused cannabis plant material may be substantially free of sticky clumps. In certain embodiments, wherein if the pre-rolled joint is taken apart, the homogeneous extract-infused cannabis biomass may fall apart easily without external force.

In certain embodiments, a homogeneous extract-infused cannabis biomass of the present disclosure may comprise a predefined amount of at least one cannabinoid, at least one terpene, or any combination thereof, wherein at least a portion of the predefined amount of the at least one cannabinoid, the at least one terpene, or combination thereof may be from an exogenous source to an originating cannabis plant material, and wherein there may be less than 15% variability in concentration the at least one cannabinoid, at least one terpene, or combination thereof across any given mass of the homogeneous extract-infused cannabis biomass.

In certain embodiments, the exogenous source may be a cannabis extract. In certain embodiments, the exogenous source may be a cannabis distillate or a cannabis concentrate.

In certain embodiments, there is provided a method of preparing a pre-rolled joint, the method comprising providing a homogeneous extract-infused biomass prepared according to a method described herein; freezing or cooling the extract-infused cannabis biomass to produce a frozen extract-infused cannabis biomass; providing the frozen extract-infused cannabis biomass to a filling device and forming a pre-rolled smokable product comprising the frozen extract-infused cannabis product, wherein the filling device may be configured to provide the pre-rolled smokable product; and tamping the pre-rolled smokable product to a desired packing density to produce the homogeneous extract-infused cannabis pre-rolled joint.

Other aspects and embodiments of the disclosure are evident in view of the detailed description provided herein.

### BRIEF DESCRIPTION OF THE DRAWINGS

In drawings which illustrate non-limiting embodiments of the present disclosure:

FIG. 1 is a photographic image of an exemplary liquid extract-infused cannabis biomass from an extract-infused cannabis product (pre-rolled joint) produced using conventional techniques. Panel A shows the extract-infused cannabis plant material of the product. Panel B is an image of a pre-rolled joint containing the extract-infused cannabis plant material of panel A. A black arrow indicates a region of dark plant material in panel A and a black arrow indicates a region with oil-like residue in panel B;

FIG. 2 is a photographic image of an exemplary liquid extract-infused cannabis plant material from an extract-infused cannabis product (pre-rolled joint) as described herein. Panel A is an image of a pre-rolled joint containing the extract-infused cannabis plant material. Panel B shows the extract-infused cannabis plant material of the product of panel A;

FIG. 3 is a photographic image of another exemplary liquid extract-infused cannabis biomass from an extract-infused cannabis product (pre-rolled joint) produced using conventional techniques. Panel A shows the extract-infused cannabis plant material of the product. Panel B is an image of a pre-rolled joint containing the extract-infused cannabis plant material of panel A. A white arrow indicates a region of dark plant material in panel A and a black arrow indicates a region with oil-like residue in panel B

FIG. 4 is a photographic image of an exemplary liquid extract-infused cannabis plant material from another extract-infused cannabis product (pre-rolled joint) as described herein. Panel A is an image of a pre-rolled joint containing the extract-infused cannabis plant material. Panel B shows the extract-infused cannabis plant material of the product of panel A;

FIG. 5 is a flow diagram of an embodiment of a method as described herein;

FIG. 6 is a flow diagram of an embodiment of a method as described herein, wherein the steps of applying and mixing are performed simultaneously;

FIG. 7 is a flow diagram of an embodiment of a method as described herein, wherein one or more additive is provided;

FIG. 8 is a flow diagram of an embodiment of a method as described herein, wherein a step of screening is performed after the mixing and wherein non-homogeneous extract-infused cannabis biomass may be further mixed one or more times to provide the homogeneous extract-infused cannabis biomass;

FIG. 9 is a flow diagram of an embodiment of a method as described herein, wherein the homogeneous extract-infused cannabis biomass is frozen, provided to a filling device and tamped to a desired density to produce a homogeneous extract-infused cannabis pre-rolled joint; and

FIG. 10 provides an embodiment of a filling device used to provide pre-rolled products.

### DETAILED DESCRIPTION

Described herein are extract-infused cannabis biomasses, extract-infused cannabis products (e.g. pre-rolled joints) and methods for preparation thereof. It will be appreciated that embodiments and examples are provided herein for illustrative purposes intended for those skilled in the art, and are not meant to be limiting in any way.

As used herein, “cannabis” may encompass any material obtained or produced from a cannabis and/or a hemp plant, such as for example, Cannabis ruderalis, Cannabis indica, and Cannabis sativa.

Cannabis plants produce a diverse array of secondary metabolites, including cannabinoids, terpenes and terpenoids, sterols, triglycerides, alkanes, squalenes, tocopherols, carotenoids and alkaloids. The combination and levels of these secondary metabolites varies depending on several factors, including, but not limited to, the cannabis variety, the part of the cannabis plant extracted, the method of extraction, the processing of the extract, and the season. The skilled person having regard to the teachings herein will be able to select factors that may produce different secondary metabolites, such as cannabinoids and/or terpenes, for a given application. In addition, as would be known by the person of skill, the level of secondary metabolites, as well as their specific combinations may produce distinct physiological outcomes on the user if they consume cannabis.

As used herein, “predefined” is intended to refer to a known or determined amount. For example, predefined may be a known concentration or amount of a particular cannabinoid or terpene in a given cannabis extract or particular strain of cannabis plant (or a plant material of that plant). In other embodiments, predefined may be an experimentally determined concentration or amount of a particular cannabinoid or terpene from a given source.

In certain embodiments described herein, it is contemplated that the predefined amount or concentration may be a known value used to provide a product or perform a method as described herein that produces a homogeneous target concentration of at least one cannabinoid and/or at least one terpene in the product or biomass. In certain embodiments described herein, it is contemplated that the predefined amount or concentration may be calculated to provide a product or biomass or perform a method as described herein that produces a homogeneous desired target concentration of at least one cannabinoid and/or at least one terpene in the product or biomass. In certain embodiments, the predefined value may be predetermined. In certain embodiments, the predefined concentration may be determined in each instance. In certain embodiments, the predefined concentration may be a known quantity, such as, for example, a quantity based on known characteristics of the extract or the plant.

In contrast to the term “predefined”, the term “target” as used herein is intended to refer to a desired concentration or amount of any given cannabinoid or terpene. The predefined concentrations or amounts are used to obtain the target concentrations or amounts in accordance with the methods disclosed herein, and resultant products. This is accomplished using a predefined ratio of cannabis plant material, cannabis extract, and optionally additive cannabinoid or additive terpene as described herein.

As used herein, “cannabinoid” may encompass any chemical compound that acts upon a cannabinoid receptor. Cannabinoids may include phytocannabinoids, which are found in cannabis and other plants. Cannabinoids may also include synthetic cannabinoids, which are artificially manufactured.

Cannabinoids may include, but are not limited to, tetrahydrocannabinol (THC), tetrahydrocannabiorcol (THCC), Δ9-THC, Δ8-THC, cannabidiol (CBD), cannabigerol (CBG), cannabichromene (CBC), cannabinol (CBN), cannabicyclol (CBL), cannabielsoin (CBE), cannabivarin (CBV), cannabifuran (CBF), cannabitran (CBT), cannabiripsol (CBR), including acidic forms thereof (e.g. THCA, CBDA, CBGA, CBCA, etc.), as well as forms of neutral or acidic cannabinoids (THCV, THCVA, CBDV, CBVA, CBGV, CBGVA, CBCV, CBCVA, etc.). In certain embodiments, the cannabinoid is THC, THCA, CBG or CBGA.

In the context of the present disclosure, reference is made to “extract-derived” cannabinoid, “plant-derived” cannabinoid, and “biomass” cannabinoid. As used herein, reference to an “extract-derived cannabinoid” is intended to refer to any amount or quantity of a given cannabinoid that originates from a cannabis extract. As used herein, reference to a “plant-derived cannabinoid” is intended to any amount or quantity of a given cannabinoid present in and/or native to the plant material used in the methods and products herein. As used herein, “biomass cannabinoid” is intended to refer to the combined amount of the extract-derived cannabinoid, the plant-derived cannabinoid, and optionally any additive cannabinoid, present in the resultant biomass from the methods herein and/or the biomass as used in the products herein (e.g. pre-rolled joints). As will be understood, if either the cannabis extract or the plant material does not have any of a given cannabinoid, and/or an additive cannabinoid is not used, then the biomass cannabinoid is equivalent to the quantity of the given cannabinoid in the source (extract, plant or additive) that does include the cannabinoid.

The term “terpene”, as used herein, is intended to encompass both terpenes and terpenoids. Terpenes refer to a class of hydrocarbon molecules, which may provide a unique smell and may have physiological effects on the user, wherein said physiological effects may include, but are not limited to, changes in the mood, appetite, respiration, heart rate, memory, learning, perception, problem solving, coordination, heart rate, or anxiety. Terpenoids are terpene compounds that have been further metabolised in the plant, typically through an oxidative process, and therefore usually contain at least one oxygen atom.

Terpenes may include, but are not limited to, bisabolol, borneol, camphene, carene, caryophyllene, eucalyptol, geraniol, humulene, limonene, linalool, myrcene, nerolido, pinene, terpineol, and valencene. Terpenoids may include, but are not limited to, any hemiterpenoids, monoterpenoid, sesquiterpenoid, diterpenoid, sesterterpenoid, triterpenoid, tetraterpenoid or polyterpenoid. Common terpenoids include, without limitation, citral, menthol, camphor, salvinorin A, and ginkgolide.

In the context of the present disclosure, reference is made to “extract-derived” terpene, “plant-derived” terpene, and “biomass” terpene. As used herein, reference to an “extract-derived terpene” is intended to refer to any amount or quantity of a given terpene that originates from a cannabis extract. As used herein, reference to a “plant-derived terpene” is intended to any amount or quantity of a given terpene present in and/or native to the plant material used in the methods and products herein. As used herein, “biomass terpene” is intended to refer to the combined amount of the extract-derived terpene, the plant-derived terpene, and optionally any additive terpene, present in the resultant biomass from the methods herein and/or the biomass as used in the products herein (e.g. pre-rolled joints). As will be understood, if either the cannabis extract or the plant material does not have any of the given terpene, and/or an additive terpene is not used, then the biomass terpene is equivalent to the quantity of the given terpene in the source (extract, plant or additive) that does include the terpene.

As used herein, “cannabis extract” may refer to a cannabis material that contains higher levels of secondary metabolites, such as for example, cannabinoids and/or terpenes, than found in a cannabis plant material to which it was derived from. In certain embodiments, the cannabis extract may be a liquid or may be capable of conversion to a liquid-like consistency. In certain embodiments, the liquid cannabis extract may comprise a resin, live resin, rosin, live rosin, sauce, crystalline/diamond, concentrate, distillate, isolate, hash, kief, terpenes, botanicals, or any combination thereof. In certain embodiments, the cannabis extract is a cannabis distillate. As used herein, a “cannabis distillate” is intended to refer to a cannabis extract isolated or obtained using a distillation procedure. In an embodiment, the cannabis distillate does not retain a substantial amount of the natural terpenes found in the strain of cannabis plant material in which the cannabis distillate was extracted. In certain embodiments, the cannabis extract is a cannabis concentrate. As used herein, a cannabis concentrate may be a cannabis extract that retains some of the natural terpenes present in the strain of cannabis plant material in which the cannabis concentrate was extracted or has additive terpene. In certain embodiments, the cannabis plant material may comprise a homogeneous extract-infused cannabis biomass or an extract-infused cannabis biomass. In certain embodiments, a homogeneous extract-infused cannabis biomass or an extract-infused cannabis biomass may be infused with a cannabis extract.

Typically, the use of a liquid cannabis extract, such as a cannabis distillate or cannabis concentrate (e.g. liquefied or flowable), presents challenges in the production of extract-infused cannabis products. Specifically, unlike solid-extracts, the use of liquid extracts may require additional unique steps, such as, for example, applying and mixing which may not be applicable to the production of extract-infused products made using solid extracts. A person of skill in the art having regard to the teachings herein would recognize that the steps such as applying and mixing may be important features of the method or product, which provide a target concentration of at least one cannabinoid and/or at least one terpene, which may result in a specific outcome for the user. Additionally, the person of skill in the art having regard to the teachings herein would be aware that changes in cannabinoid amount and/or terpene amount may alter the properties of the cannabis extract. Cannabis extract with an increased concentration of terpenes may require different parameters in production, such as temperature, type of applying, or type of mixing required to obtain a homogeneous extract-infused cannabis product, when compared to those with an increased concentration of cannabinoids.

A person of skill in the art will recognize that different methods to prepare a cannabis extract may be possible and said methods may alter aspects of the cannabis extract. The skilled person having regard to the teachings herein will be able to select a suitable method for a given application or select a suitable cannabis extract prepared using a suitable method for a given application. Any suitable extractant may be used, including, for example, alcohols (for example, methanol, ethanol, propanol, butanol, or propylene glycol), water, hydrocarbons (for example, butane, or hexane), oils (for example, olive oil, vegetable oil, or essential oils), a polar organic solvent (for example, ethyl acetate, or polyethylene glycol) or a supercritical fluid (for example, supercritical CO₂). The extractant may be completely or partially removed prior to providing the cannabis extract, or it may be included in the cannabis extract. In certain embodiments, the cannabis extract selected for use contains minimal or no residual traces of any solvent used in the extraction process. As used herein “minimal or no residual traces of any solvent used in the extraction process”, refers to solvent levels for example within the tolerance limits referred to in subsection 93(3) or 94(2) or section 101.1 of the Canadian Cannabis Regulations.

As used herein, the term “cannabis product” or “product” may encompass any vendible item comprising cannabis, such as, for example, a pre-rolled joint. Pre-rolled joints of the present disclosure comprise the homogeneous extract-infused biomass as disclosed herein, wrapped into the form of a cigarette. In certain embodiments, the cannabis product or product may comprise a label and a closure system. In further embodiments, the label and the closure system may comply with local regulations for the sale and distribution of cannabis and cannabis related products.

As used herein, the term “homogeneous” is intended to mean consisting of parts that are substantially identical, the same or alike throughout. In certain embodiments, the term “homogeneous” may refer to a feature, such as a cannabinoid and/or terpene concentration. Thus, in an embodiment, the term homogenous as used herein means a substantially consistent or identical concentration or amount of a particular cannabinoid and/or terpene throughout the product (e.g. biomass and/or pre-rolled joint). In an embodiment, by substantially consistent or identical, it is meant that the feature (e.g. cannabinoid concentration) exhibits less than 15% variability throughout. In an embodiment, the term homogeneous may refer to a feature that exhibits less than 10% variability throughout, or even less as described elsewhere herein.

As used herein, the term “extract-infused” refers to the introduction of a cannabis extract into or onto another element, such as a cannabis plant material to produce an extract-infused cannabis plant material. In certain embodiments herein, a homogeneous extract-infused cannabis biomass or homogeneous extract-infused cannabis product may refer to a substantially identical ratio, concentration or amount of a cannabis plant material and a cannabis extract throughout a prepared batch, alternative batches, a product, or any combination thereof. In certain embodiments, the homogeneous extract-infused cannabis biomass may comprise at least 55% [w/w], at least 60% [w/w], at least 70% [w/w], or at least 75% [w/w], of the cannabis plant material. A ratio that provides too much extract may not facilitate performing the methods or the products described herein, as the increased amount of the cannabis extract may result in an extract-infused plant material that is unable to be mixed, is not homogeneous, or is not amenable to downstream steps for product production, such as the production of a pre-rolled joint. A ratio that does not provide enough extract may not be homogeneous as the volume may be insufficient to apply to the entire cannabis plant material provided with the cannabis extract and/or may result in physical properties that are not amenable to product formation.

In certain embodiments herein, a homogeneous extract-infused cannabis biomass or homogeneous extract-infused cannabis product may refer to a substantially identical cannabinoid and/or terpene amount, and/or a substantially identical cannabinoid and/or terpene concentration throughout a prepared batch, alternative batches, a product, or any combination thereof. In certain embodiments, the homogeneous extract-infused cannabis biomass or the homogeneous extract-infused cannabis product may have a measured cannabinoid concentration and/or a measured terpene concentration that is equal, within at least 15%, within at least 10%, within at least 7.5%, within at least 5%, within at least 4%, between about 1% and about 10%, or between about 3% and about 7% variability throughout a prepared batch, alternative batches, a product, or any combination thereof. The skilled person, in light of the teachings herein, would appreciate that a measured concentration, such as a measured cannabinoid and/or terpene concentration, with a lower variability is more homogeneous than that of a prepared batch, alternative batches, a product, or any combination thereof with a higher variability.

Unlike the homogenous products of the present disclosure, a non-uniform or heterogeneous distribution of a cannabis extract on a cannabis plant material results in a product with a substantially uneven distribution of the at least one cannabinoid and/or the at least one terpene. Uneven distribution of the at least one cannabinoid and/or the at least one terpene may result in loss of consumable cannabinoid and/or terpene, as the oversaturated regions may not burn optimally, decrease the activation of cannabinoids, and/or cause adverse reactions due to the elevated amount of cannabinoid and/or terpene within a specific region or regions of the product. A person of skill would appreciate, in light of the teachings herein, that a portion of cannabis plant material oversaturated with extract may result in an oily residue leaching through to the external surface of the wrap or paper, which may compromise the structural integrity of the product. A person of skill in the art would appreciate that an over-saturated cannabis plant material may produce a product, such as a pre-rolled joint, with limited structural integrity compared to a pre-rolled joint produced with a homogeneously infused extract. A joint with limited structural integrity may break while being consumed which may result in a loss of product.

Unlike the methods of the present disclosure, existing methods, such as injection of a liquid extract, as would be known to the person of skill having regard to the teachings of Custom Cones USA (https://customconesusa.com/automated-infused-pre-roll-machine/), may produce products, such as pre-rolled joints, with a substantially heterogeneous distribution of extract. As would be known to the skilled person, the injection of liquid extract produces regions of high extract amount, where as other regions may have a lower extract amount or be devoid of extract. The technology of Custom Cones USA provides a single injection needle which penetrates the core or the centre of the cannabis plant material already present in a pre-rolled joint to provide the cannabis extract-infused product. As such, extract-infused cannabis products made using conventional techniques are heterogeneous and thus may not meet local regulatory standards and/or consistently meet local regulatory standards.

As used herein, “derived” is intended to mean obtained or taken from. In certain embodiments herein, cannabinoids and/or terpenes may be derived from cannabinoids and/or terpenes in a cannabis plant material, a cannabis extract, an additive, or a biomass, and may be referred to as plant-derived, extract-derived, additive-derived, and biomass-derived, respectively.

Exemplary Methods and Products thereof of the Present Disclosure

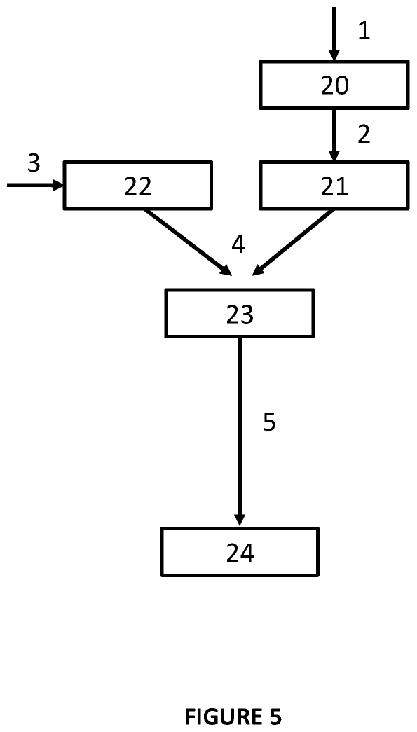

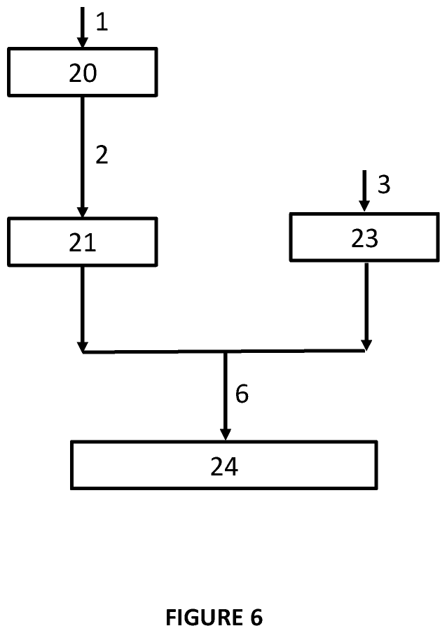

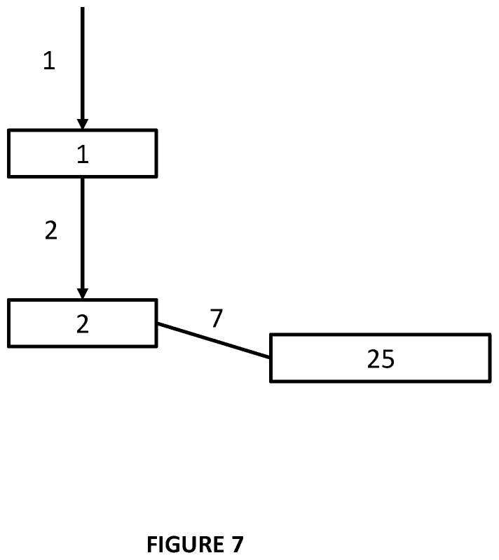

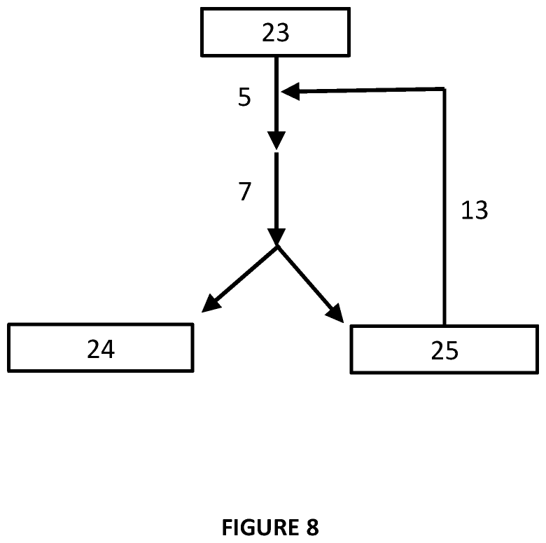

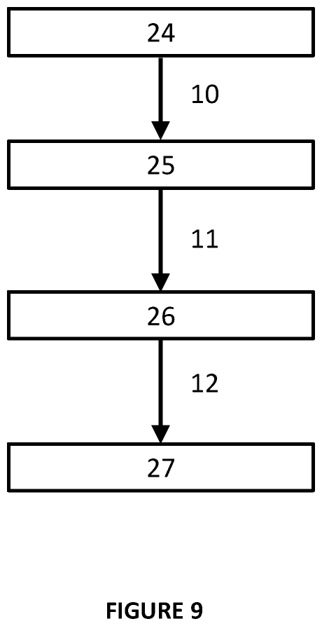

An example of methods for preparing a homogeneous extract-infused cannabis biomass (24) and/or product as described herein is depicted in FIGS. 5-10. With reference to FIGS. 5-10, the depicted methods comprise: providing (1) a cannabis extract 20 having a predefined concentration of at least one extract-derived cannabinoid, at least one extract-derived terpene, or any combination thereof; heating (2) the cannabis extract 20 at a temperature of up to 120° C. to provide a heated cannabis extract 21, wherein the heated cannabis extract 21 may be in a substantially liquid or flowable state; providing (3) a cannabis plant material 22 having a predefined concentration of at least one plant-derived cannabinoid, at least one plant-derived terpene, or any combination thereof, wherein the cannabis plant material 22 is a milled or comminuted cannabis plant material; applying (4) the heated cannabis extract 21 to the cannabis plant material 22 using a predefined ratio of the cannabis plant material 22 to the heated cannabis extract to provide an extract applied plant material 23, wherein the predefined ratio provides a target amount of at least one biomass cannabinoid, at least one biomass terpene, or any combination thereof within the extract-applied plant material; and mixing (5) the extract-applied plant material to provide a homogeneous extract-infused cannabis biomass 24, wherein there is less than a 15% variability in a target concentration of the at least one biomass cannabinoid, the at least one biomass terpene, or any combination thereof throughout a prepared batch and/or between alternate batches of the homogeneous extract infused cannabis biomass.

The methods herein comprise a step of providing (1) a cannabis extract having a predefined concentration of at least one extract-derived cannabinoid, at least one extract-derived terpene, or any combination thereof. In certain embodiments, the step of providing the cannabis extract may comprise providing a previously obtained cannabis extract. For example, the cannabis extract may be purchased from a third party source, may be extracted from plant material at an alternate site, may be stored until needed, or any combination thereof. In certain embodiments, the step of providing a cannabis extract may comprise extracting the cannabis extract from a cannabis plant material. In an embodiment, the cannabis extract is a cannabis concentrate, a cannabis distillate, a cannabis oil, a cannabis isolate, a cannabis resin, or any combination thereof. In a preferred embodiment, the cannabis extract is a cannabis distillate.

In an embodiment, the cannabis extract is a single type of cannabis extract (e.g. a cannabis distillate). In another embodiment, two or more cannabis extracts may be used. For example, in an embodiment the cannabis extract is any two or more of a cannabis distillate, a cannabis concentrate, and a cannabis isolate. In an embodiment, the cannabis extract is a cannabis concentrate and a cannabis isolate. In another embodiment, the cannabis extract is a cannabis distillate and a cannabis isolate. When two or more cannabis extracts are used, they may be mixed together prior to applying to the cannabis plant material or they may be applied to the cannabis plant material separately, either at the same time or at alternate times.

The method herein comprises a step of heating (2) the cannabis extract at a temperature of up to 120° C. to provide a heated cannabis extract. In certain embodiments, the step of heating may be performed by heating the cannabis extract to a temperature of up to 120° C. In certain embodiments, the step of heating may be performed by heating the cannabis extract at or to a temperature of between about 60° C. and about 120° C. In certain embodiments, the step of heating may be performed by heating the cannabis extract at or to a temperature of up to 60° C., up to 61° C., up to 62° C., up to 63° C., up to 64° C., up to 65° C., up to 66° C., up to 67° C., up to 68° C., up to 69° C., up to 70° C., up to 71° C., up to 72° C., up to 73° C., up to 74° C., up to 75° C., up to 76° C., up to 77° C., up to 78° C., up to 79° C., up to 80° C., up to 81° C., up to 82° C., up to 83° C., up to 84° C., up to 85° C., up to 86° C., up to 87° C., up to 88° C., up to 89° C., up to 90° C., up to 91° C., up to 92° C., up to 93° C., up to 94° C., up to 95° C., up to 96° C., up to 97° C., up to 98° C., up to 99° C., up to 100° C., up to 101° C., up to 102° C., up to 103° C., up to 104° C., up to 105° C., up to 106° C., up to 107° C., up to 108° C., up to 109° C., up to 110° C., up to 111° C., up to 112° C., up to 113° C., up to 114° C., up to 115° C., up to 116° C., up to 117° C., up to 118° C., up to 119° C., up to 120° C., up to 121° C., up to 122° C., up to 123° C., up to 124° C., up to 125° C., up to 126° C., up to 127° C., up to 128° C., up to 129° C., up to 130° C., up to 131° C., up to 132° C., up to 133° C., up to 134° C., up to 135° C., up to 136° C., up to 137° C., up to 138° C., up to 139° C., or up to 140° C. In certain embodiments, the cannabis extract may be provided to a stainless-steel vessel and heated. In certain embodiments, the step of heating is performed using a conventional heating element.

In certain embodiments, the step of heating may be performed by heating the cannabis extract at or to a temperature of about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., or about 120° C.

In certain embodiments, the a step of heating the cannabis extract at a temperature of up to 120° C. to provide a heated cannabis extract may be performed at a temperature and for a duration to produce a substantially liquid or flowable cannabis extract. In certain embodiments, the duration of the heating may be determined using the equation:

$$The length of time for heating of the cannabis extract ( s ) = \frac{m \times cp \times Δ T}{P}$$

wherein m is a mass of the cannabis extract, cp is a specific heat capacity of the cannabis extract in J/kg° C., ΔT is a temperature change (° C.) of the cannabis extract; and P is a rate of energy (W) transfer to the cannabis extract. In certain embodiments, the cannabis extract may be heated for between about 1 min and about 600 min. In certain embodiments, the cannabis extract may be heated for about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, or about 90 min. The skilled person, in light of the teachings herein, would be able to select an appropriate method for heating the cannabis extract at a temperature of up to 120° C. to provide the heated cannabis extract. In certain embodiments, the step of heating may comprise an additional step of stirring the heating cannabis extract. In certain embodiments, the stirring the heating cannabis extract may comprise manual stirring or stirring using magnetic means, such as, for example, a magnetic stir bar. In certain embodiments, the heating cannabis extract may be stirred for a period of 1 minute every other 10 minutes during the step of heating.

As disclosed in the Examples herein, heating temperatures of approximately 50° C. to 100° C. were advantageous in obtaining a homogeneous extract-infused cannabis biomass and/or a homogeneous extract-infused cannabis product. Heating the cannabis extract with temperatures of approximately 40° C. were less effective. Heating the cannabis extract with temperatures of approximately 40° C. resulted in inconsistent particle size, and often a non-homogeneous extract-infused cannabis biomass.

As used herein, the expressions “substantially liquid” or “flowable state” may refer to a viscosity of the cannabis extract or the heated cannabis extract such that the material properties of the cannabis extract permit the application of the heated cannabis extract to the cannabis plant material. The skilled person, in light of the teachings herein, would be able to select an appropriate temperature to provide a substantially liquid cannabis extract. The use of a cannabis extract that is not substantially liquid may result in an inability to perform the methods as described herein. The skilled person, in light of the teachings herein, would also be able to select an appropriate temperature for the heating that does not degrade any cannabinoids and/or terpenes in the cannabis extract. In certain embodiments, extract-derived cannabinoid, extract-derived terpene, additive-derived cannabinoid, additive-derived terpene, or any combination thereof may be in any state of matter, such as for example a liquid or solid, prior to their inclusion into a cannabis extract.

The methods herein comprise a step of providing (3) a cannabis plant material having a predefined concentration of at least one plant-derived cannabinoid, at least one plant-derived terpene, or any combination thereof. The plant material may be any suitable part of a cannabis plant, including for example whole plant, buds, flower, flower portions, bark, rhizomes, fruit, or any combination thereof. In an embodiment, the plant material is bud or flower. In an embodiment, the plant material is dried flower. The plant material (e.g. flower) may be dried by any suitable and/or conventional means. In certain embodiments, the step of providing the cannabis extract may comprise providing a previously obtained or prepared plant material. For example, the cannabis plant material may be purchased from a third party source, may be dried or otherwise prepared by a third party, may be stored until needed, or any combination thereof. In certain embodiments, the step of providing a cannabis plant material comprises any one or more steps of cultivating cannabis plants, harvesting the desired part of the cannabis plant (e.g. flower), drying the plant material from the plant, or any combination thereof.

In certain embodiments, the cannabis plant material may be a milled or a comminuted cannabis plant material. As used herein, “milling” or “comminuting” may refer to a reduction in size of a cannabis plant material from its size following harvesting, its size prior to processing and/or its size before removal from the plant. In certain embodiments, the cannabis plant material may be milled or comminuted between about 0.5 mm and about 10 mm. In certain embodiments, the cannabis plant material may be milled or comminuted between about 1 mm and about 5.0 mm. In certain embodiments, the cannabis plant material may be milled or comminuted between about 1 mm and about 3.5 mm. In certain embodiments, the cannabis plant material may be milled or comminuted to a particle size of less than or equal to 5.0 mm. In certain embodiments, the cannabis plant material may be milled or comminuted to a particle size between about 100 μm and about 3.5 mm.

Having regard to the present disclosure, the particle size of the cannabis plant material may impact the quality of the homogeneous extract-infused biomass or to the ability to perform a method as described herein, for example from the perspective of one or more of the following: extract absorption by the cannabis plant material, extract retention by the cannabis plant material, ability to perform the method, ability to form the product, stability of the product, consumption of the product, including burning, and the desired outcome for the user. In certain embodiments, the cannabis plant material may be milled or comminuted using milling and/or grinding techniques known to the skilled person. In certain embodiments, the cannabis plant material may be milled using a grinder, wherein the grinder may be set at between about 20 and about 60 revolutions per minute and coupled to a screen with a perforation size of 3.2 mm.

In an embodiment, the cannabis plant material is a single type or source of cannabis plant (e.g. a single cannabis plant strain). In another embodiment, two or more different cannabis plant materials (e.g. strains) may be used. When two or more cannabis plant materials are used, they are typically mixed together before applying the cannabis extract. However, it is contemplated that the cannabis extract may be applied to each plant material separately and then the plant materials combined.

The methods herein involve a step of applying (4) the heated cannabis extract to the cannabis plant material. In an embodiment, the applying comprises pouring, spraying, misting, sprinkling, showering, diffusing, or kneading. In a particular embodiment, the applying comprises a step of mixing in the form of kneading. In the context of the methods herein, the step of applying is performed in a manner such that it uniformly disperses the cannabis extract on the cannabis plant material. This is achieved through a combination of features of the methods of the present disclosure, including without limitation appropriate heating of the cannabis extract, milling of the plant material, and manner of application. Unlike the methods disclosed herein, in instances where cannabis extract is not uniformly dispersed on the cannabis plant material, the cannabinoid concentration and/or terpene concentration is not uniform across the batch (e.g. >15% variability), and there is often significant variability between batches (i.e. lack of reproducibility). Moreover, if the cannabis extract is not uniformly dispersed on the cannabis plant material, the resulting mixture may form structures, such as ball-like structures, which may not be amenable to subsequent method steps or may not be amenable for desired applications, such as forming a smokable extract-infused cannabis product (e.g. pre-rolled joint). By way of non-limiting example, it was found that heating the cannabis extract to 40° C. results in a non-homogeneous extract-infused cannabis biomass that may form ball-like structures.

In accordance with the methods herein, the heated cannabis extract is applied (4) to the cannabis plant material using a predefined ratio of the cannabis plant material to the heated cannabis extract to provide an extract applied plant material. The predefined ratio of the cannabis plant material to the heated cannabis extract, optionally taking into account any additive cannabinoid and/or additive terpene that may be used, provides the target amount of the at least one biomass cannabinoid and/or at least one biomass terpene within the extract-applied plant material. By “target amount”, it is intended to mean the total amount of a given cannabinoid and/or terpene within the biomass. In the context of the methods and products herein, this target amount of the given cannabinoid and/or terpene is evenly distributed throughout the biomass to then provide the homogeneous extract-infused cannabis biomass with less than a 15% variability in target concentration of the given cannabinoid and/or terpene.

In an embodiment, the target amount is of a particular cannabinoid (e.g. THC or CBD), such that the predefined ratio is a calculated ratio of cannabis plant material to cannabis extract to provide that target amount of that cannabinoid in the biomass (i.e. biomass cannabinoid), optionally with the addition of additive cannabinoid. In some embodiments, the target amount comes from both the cannabis plant material and cannabis extract. In some embodiments, the target amount comes from only one of the cannabis plant material or the cannabis extract. In some embodiments, the target amount comes from one or both of the cannabis plant material and the cannabis extract, and the additive cannabinoid.

In an embodiment, the target amount is individually and independently for two or more particular cannabinoids (e.g. THC and CBD), such that the predefined ratio is a calculated ratio of cannabis plant material to cannabis extract to provide the target amount for each of the cannabinoids in the biomass (i.e. biomass cannabinoids), optionally with the addition of additive cannabinoid for one or both or the cannabinoids. In an embodiment, two or more cannabis extracts are used when it is desired to have a target amount of two or more different cannabinoids. In some embodiments, the target amount of any particular cannabinoid comes from both the cannabis plant material and cannabis extract. In some embodiments, the target amount of any particular cannabinoid comes from only one of the cannabis plant material or the cannabis extract. In some embodiments, the target amount of any particular cannabinoid comes from one or both of the cannabis plant material or the cannabis extract, and the additive cannabinoid.

In an embodiment, the target amount is of a particular terpene (e.g. linalool), such that the predefined ratio is a calculated ratio of cannabis plant material to cannabis extract to provide that target amount of that terpene in the biomass (i.e. biomass terpene), optionally with the addition of additive terpene. In some embodiments, the target amount comes from only one of the cannabis plant material or the cannabis extract. In some embodiments, the target amount comes from one or both of the cannabis plant material and the cannabis extract, and the additive terpene.

In an embodiment, the target amount is individually and independently for two or more particular terpenes (e.g. linalool and caryophyllene), such that the predefined ratio is a calculated ratio of cannabis plant material to cannabis extract to provide the target amount for each of the terpenes in the biomass (i.e. biomass terpenes), optionally with the addition of additive terpene for one or both or the terpenes. In an embodiment, two or more plant materials are used when it is desired to have a target amount of two or more different terpenes. In some embodiments, the target amount of any particular terpene comes from both the cannabis plant material and cannabis extract. In some embodiments, the target amount of any particular terpene comes from only one of the cannabis plant material or the cannabis extract. In some embodiments, the target amount of any particular terpene comes from one or both of the cannabis plant material or the cannabis extract, and the additive terpene.

In an embodiment, the target amount is individually and independently for one or more cannabinoids and one or more terpenes, such that the predefined ratio is a calculated ratio of cannabis plant material to cannabis extract to provide the target amount for each of the cannabinoids and terpenes in the biomass (i.e. biomass cannabinoids and terpenes), optionally with the addition of additive cannabinoid and/or additive terpene for any one or more of the cannabinoids and terpenes. In an embodiment, two or more cannabis extracts, plant materials, or any combination thereof are used when it is desired to have a target amount of one or more cannabinoids and one or more terpenes. In some embodiments, the target amount of any particular cannabinoid or terpene comes from both the cannabis plant material and cannabis extract. In some embodiments, the target amount of any particular cannabinoid or terpene comes from only one of the cannabis plant material or the cannabis extract. In some embodiments, the target amount of any particular cannabinoid or terpene comes from one or both of the cannabis plant material or the cannabis extract, and the additive cannabinoid and/or additive terpene.

In certain embodiments, the predefined ratio may comprise an amount, such as a mass or volume, of the cannabis extract to an amount, such as a mass or volume, of the cannabis plant material. In an embodiment, the predefined ratio is a mass:mass ratio of the cannabis plant material to the cannabis extract. In an embodiment, the predefined ratio by weight cannabis plant material to cannabis extract is between about 100:1 and about 1:1, more particularly between about 10:1 and about 1:1, and more particularly still between about 5:1 and about 2:1. In an embodiment, the predefined ratio by weight cannabis plant material to cannabis extract is about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1. In an embodiment, the predefined ratio by weight cannabis plant material to cannabis extract is about 70:30. In an embodiment, the predefined ratio by weight cannabis plant material to cannabis extract is about 84:16. In an embodiment, the predefined ratio by weight cannabis plant material to cannabis extract is about 76:23, and in an embodiment thereof the cannabis extracts is portion is comprised two different cannabis extracts at a ratio of 19:4 (i.e. 76:19:4).

A the skilled person will appreciate having regard to the disclosure herein, the predefined ratio is dependent upon the predefined concentration of the given cannabinoid and/or terpene in the cannabis extract, the predefined concentration of the given cannabinoid and/or terpene in the cannabis plant material, and optionally the added amount of any additive cannabinoid and/or terpene used in the methods.

In certain embodiments, the cannabinoid and/or terpene concentration of the cannabis extract may be determined by weight of cannabinoid or terpene per volume or weight, or moles of cannabinoid or terpene per volume or weight. In certain embodiments, the cannabinoid and/or terpene concentration of the cannabis plant material may be determined in weight of cannabinoid or terpene per weight of cannabis plant material, or moles of cannabinoid or terpene per weight of cannabis plant material.

In certain embodiments, the calculation used to provide the homogeneous extract-infused cannabis biomass of the present disclosure, having a particular desired cannabinoid or terpene concentration may be as follows:

$$Cannabinoid or terpene concentration ( mg / g ) = \frac{[ A \times B ] + [ C \times D ]}{[ A + C ]}$$

where, A is a mass (g) of the cannabis plant material; B is total cannabinoid concentration (mg/g) or total terpene concentration (mg/g) of the cannabis plant material; C is a mass (g) of a cannabis extract; and D is cannabinoid concentration (mg/g) of the cannabis extract. In certain embodiments, the cannabis plant material forms at least 55%, at least 60%, at least 65%, at least 70%, or at least 75% of the total weight of the extract-applied plant material.

By applying a heated cannabis extract to a cannabis plant material as described herein, a known mass of heated cannabis extract may be applied to a known mass of cannabis plant material, in order to produce an extract-applied plant material having the target amount of one or more given cannabinoids and/or terpenes. In an exemplary non-limiting example, if a particular variety of high-CBD containing cannabis plant material is to be used as the cannabis plant material and if a hemp extract is to be used as the cannabis extract, an average CBD amount for one gram of the variety of high-CBD cannabis plant material is known or is determined, and the CBD amount for the specific weight or volume of cannabis extract applied to one gram of the high-CBD hemp is known or is determined. Therefore, the total cannabinoid and/or terpene amount or concentration is known or is determined, which can be provided to medical, government, and/or regulatory officials for support to provide accurate dosing. Additionally, the amount of cannabinoid and/or terpene in a particular weight of product may be increased by increasing the volume of cannabis extract applied to the cannabis plant material, increasing the concentration of the at least one cannabinoid and/or the at least one terpene provided in the cannabis extract, by providing a cannabis plant material with an increased cannabinoid and/or terpene amount or concentration, or any combination thereof. Thus, a person who needs a higher dosage of THC, for example, will not need to smoke more product but instead said person can smoke a product with a higher ratio of the cannabis extract to the cannabis plant material to achieve the desired outcome. A person of skill in the art will recognize that smoking less product, while still obtaining the desired outcome, may limit the negative health impacts when compared to smoking an increased amount of product with less cannabinoid and/or terpene to achieve the same desired outcome.

In accordance with the methods herein, the extract-applied plant material is mixed (5) to provide a homogeneous extract-infused cannabis biomass. In certain embodiments, the mixing may be by rotational mixing, blending, stirring, beating, kneading, or any combination thereof of the extract-applied plant material, whether by manual, machine operated and/or automated techniques. In certain further embodiments, the mixing is performed in a mixer comprising a flat beater attachment and the flat beater attachment mixes the cannabis plant material and the heated cannabis extract. In certain further embodiments, the flat beater attachment mixes the cannabis plant material and the heated cannabis extract at between about 80 rpm and about 120 rpm.

In an embodiment, the mixing is performed under heated conditions. In certain embodiments, the heated conditions are at a temperature of up to 120° C. In certain embodiments, the heated conditions are at a temperature of between about 60° C. and about 120° C. In certain embodiments, the heated conditions are at or to a temperature of up to 60° C., up to 61° C., up to 62° C., up to 63° C., up to 64° C., up to 65° C., up to 66° C., up to 67° C., up to 68° C., up to 69° C., up to 70° C., up to 71° C., up to 72° C., up to 73° C., up to 74° C., up to 75° C., up to 76° C., up to 77° C., up to 78° C., up to 79° C., up to 80° C., up to 81° C., up to 82° C., up to 83° C., up to 84° C., up to 85° C., up to 86° C., up to 87° C., up to 88° C., up to 89° C., up to 90° C., up to 91° C., up to 92° C., up to 93° C., up to 94° C., up to 95° C., up to 96° C., up to 97° C., up to 98° C., up to 99° C., up to 100° C., up to 101° C., up to 102° C., up to 103° C., up to 104° C., up to 105° C., up to 106° C., up to 107° C., up to 108° C., up to 109° C., up to 110° C., up to 111° C., up to 112° C., up to 113° C., up to 114° C., up to 115° C., up to 116° C., up to 117° C., up to 118° C., up to 119° C., or up to 120° C. In certain embodiments, the cannabis extract may be provided to a stainless-steel vessel and heated. In certain embodiments, the step of heating is performed using a conventional heating element.

In certain embodiments, the heated conditions are at or to a temperature of about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., or about 120° C.

In certain embodiments, a target concentration of a given cannabinoid and/or terpene may be any acceptable cannabinoid concentration and/or terpene concentration that is selected by, for example, the user, the manufacturer or the producer.

In certain embodiments, the target concentration of the given cannabinoid or terpene may be a cannabinoid concentration and/or a terpene concentration selected to achieve a specific cannabinoid amount and/or a specific terpene amount. In certain embodiments, the concentration of the at least one biomass cannabinoid and/or the at least one biomass terpene may be one to meet regulatory standards.

In certain embodiments, the target concentration of the at least one biomass cannabinoid and/or the at least one biomass terpene may be a concentration that is homogeneous across batches. As will be appreciated, it is desirable for producers and/or retailers to have access to product of consistent quality between batches. Advantageously, the methods and products herein provide such desired qualities.

In certain embodiments, the target concentration of the at least one biomass cannabinoid and/or the at least one biomass terpene may be a concentration that is homogeneous within a single batch. Homogeneity within a single batch may comprise one or more of: homogeneous within a single joint, homogeneous across joints within the same package, and homogeneous across joints from multiple packages produced from the same batch. As will be appreciated, it is desirable for producers and/or retailers to have access to product of consistent quality within the individual product (e.g. pre-rolled joint) as it leads to improved consumer satisfaction. Advantageously, the methods and products herein provide such desired qualities.

In certain embodiments, the target concentration of the at least one biomass cannabinoid may be a concentration selected to produce a desired physiological outcome in the user, said physiological outcome may comprise, but are not limited to, changes in the mood, appetite, respiration, heart rate, memory, learning, perception, problem solving, coordination, heart rate, or anxiety. A person of skill in the art will recognize that subtle changes in cannabinoid concentration and/or terpene concentration may produce distinct physiological outcomes and thus may render the product undesirable if not prepared to desired specifications with respect to cannabinoid and/or terpene concentration to achieve the desired physiological outcome.

In certain embodiments, the target concentration of the at least one biomass cannabinoid and/or the at least one biomass terpene may be a concentration selected to produce a desired flavour of the extract-infused cannabis biomass. A person of skill in the art will recognize that subtle changes in cannabinoid concentration and/or terpene concentration may produce distinct flavours and thus may render the product undesirable if not prepared to desired specifications with respect to cannabinoid and/or terpene concentration. As such, the preparation of extract-infused cannabis biomass of a target concentration of given cannabinoid and/or terpene is advantageous.

The methods and products (e.g. homogenous extract-infused cannabis biomass) of the present disclosure exhibits less than a 15% variability in a target concentration of the at least one biomass cannabinoid, the at least one biomass terpene, or any combination thereof throughout a prepared batch and/or between alternate batches of the homogeneous extract infused cannabis biomass. As used herein, “variability” is intended to refer to a measurement of consistency in amount or concentration a particular cannabinoid (e.g. biomass cannabinoid), a particular terpene (e.g. biomass terpene), or any combination thereof throughout a given mass or quantity of material (e.g. cannabis biomass). In an embodiment, the given mass or quantity of material is a single batch of homogenous extract-infused cannabis biomass of the present disclosure. In an embodiment, the given mass or quantity of material is of multiple different batches of homogenous extract-infused cannabis biomass of the present disclosure. In an embodiment, the given mass or quantity of material is the biomass material within a single pre-rolled joint containing the homogenous extract-infused cannabis biomass of the present disclosure. Without being bound by theory, it is believed the reliability and reproducibility in achieving the target concentration throughout a prepared batch and between batches is in view of improvements in distribution of the cannabinoids and terpenes during infusion into or onto the plant materials.

In certain embodiments, there is between about 1% and about 15% variability in the target concentration of the at least one biomass cannabinoid, the at least one biomass terpene, or any combination thereof throughout the prepared batch, between the alternate batches, or both. In certain embodiments, there is between about 3% and about 12% variability in the target concentration of the at least one biomass cannabinoid, the at least one biomass terpene, or any combination thereof throughout the prepared batch, between the alternate batches, or both. In certain embodiments, there is between about 5% and about 10% variability in the target concentration of the at least one biomass cannabinoid, the at least one biomass terpene, or any combination thereof throughout the prepared batch, between the alternate batches, or both. In certain embodiments, there is between about 3% and about 7% variability in the target concentration of the at least one biomass cannabinoid, the at least one biomass terpene, or any combination thereof throughout the prepared batch, between the alternate batches, or both.

In certain embodiments, there is less than a 14.5% variability, less than a 14% variability, less than a 13.5% variability, less than a 13% variability, less than a 12.5% variability, less than a 12% variability, less than a 11.5% variability, less than a 11% variability, less than a 10.5% variability, less than a 10% variability, less than a 9.5% variability, less than a 9% variability, less than a 8.5% variability, less than a 8% variability, less than a 7.5% variability, less than an 8% variability, less than a 7.5% variability, less than a 7% variability, less than a 6.5% variability, less than a 6% variability, less than a 5.5% variability, less than a 5% variability, less than a 4.5% variability, less than a 4.0% variability, less than a 3.5% variability, less than a 3.0% variability, less than a 2.5% variability, less than a 2% variability, less than a 1.5% variability, less than a 1% variability, less than a 0.5% variability in the target concentration of the at least one biomass cannabinoid, the at least one biomass terpene, or any combination thereof throughout the prepared batch, between the alternate batches, or both.

In certain embodiments, there is about 14.5% variability, about 14% variability, about 13.5% variability, about 13% variability, about 12.5% variability, about 12% variability, about 11.5% variability, about 11% variability, about 10.5% variability, about 10% variability, about 9.5% variability, about 9% variability, about 8.5% variability, about 8% variability, about 7.5% variability, about 8% variability, about 7.5% variability, about 7% variability, about 6.5% variability, about 6% variability, about 5.5% variability, about 5% variability, about 4.5% variability, about 4.0% variability, about 3.5% variability, about 3.0% variability, about 2.5% variability, about 2% variability, about 1.5% variability, about 1% variability, about 0.5% variability in the target concentration of the at least one biomass cannabinoid, the at least one biomass terpene, or any combination thereof throughout the prepared batch, between the alternate batches, or both. In certain embodiments, there may be between about 0.5% to about 10%, between about 3% to about 7%, between about 0.5% and about 15%, or between about 1% to about 6% variability in the target concentration of the at least one biomass cannabinoid, the at least one biomass terpene, or any combination thereof throughout the prepared batch, between the alternate batches, or both.

A person of skill would appreciate that, in light of the teachings herein, the ability to more accurately control the concentration of a given cannabinoids and/or terpene in an extract-infused cannabis product, such as, for example less than 15% variability in a target concentration of the at least one biomass cannabinoid and/or the at least one biomass terpene, may provide the ability to provide additional exogenous cannabinoids and/or terpenes, that may not be available using conventional techniques. The person of skill would appreciate that higher variations, such as greater than 15% variability in at least one cannabinoid and/or at least one terpene concentration may not be amenable to adding additional cannabinoids and/or terpenes, as their concentration may already be too close to government variability requirements.

In certain embodiments, the variability in target concentration of the at least one biomass cannabinoid, at least one biomass terpene, or any combination thereof may be a variability throughout a prepared batch and/or between alternate batches of the homogeneous extract infused cannabis biomass. A used herein, a “batch” may refer to a quantity or amount of homogeneous extract-infused cannabis biomass or product produced at one time. In certain embodiments, an alternate batch may refer to a quantity or amount of homogeneous extract-infused cannabis biomass or product produced at different times or separately. The skilled person, in light of the teachings herein, would be able to select an appropriate method to determine or quantify the concentration of the at least one biomass cannabinoid and/or the at least one biomass terpene to determine the variability, such as, but not limited to, chromatography such as high-performance liquid chromatography (HPLC) or mass spectrometry such as gas chromatography-mass spectrometry (GC-MS). The skilled person would be able to select an appropriate number of samples, such as greater than 2, from a batch or alternate batches to test the variability in the at least one cannabinoid and/or the at least one terpene.

In certain embodiments, the steps of applying and mixing may be performed simultaneously (6). In certain embodiments, the step of applying and mixing (6) may comprise applying the heated cannabis extract to the cannabis plant material while simultaneously mixing the heated cannabis extract with the cannabis plant material. In certain embodiments, the cannabis plant material may be at room temperature prior to and during the step of applying (4), mixing (5) or the applying and mixing (6). In certain embodiments, the cannabis plant material may be at between about 50° C. and about 100° C. prior to and during the step of applying, mixing (5) or the applying and mixing (6).

In certain embodiments, the method may further comprise a step of providing (7) at least one additive (25) to the cannabis extract (20) or the heated cannabis extract (21) prior to the applying of the heated cannabis extract to the cannabis plant material. The additive may be any of a variety of ingredients known to a person of skill that may augment or may complement the cannabis extract, the extract mixture, and/or that augments or complements the target concentration of the at least one cannabinoid and/or the at least one terpene, such as providing a desired physiological outcome. The additive may improve the look or presentation, the taste or flavor, the smell, the mouth feel, the ease of application, the ability to smoke a smokable product, or other characteristic of the cannabis extract, the cannabis plant material, the homogeneous extract-infused cannabis biomass, the homogeneous extract-infused cannabis product or any combination thereof.

In an embodiment, the additive is a flavor enhancing material that may be used, such as, for example a botanical terpene blend or a cannabis derived terpene blend.

In an embodiment, the additive may be a terpene or a cannabinoid. In an embodiment, the added cannabinoid and/or terpene may be different than the cannabinoid or terpene for which a target concentration in the extract-infused cannabis biomass is desired (i.e. the biomass cannabinoid or biomass terpene). Cannabinoids may be formulated in such a way to create new effects and or increased effects not found within naturally occurring cannabis, such as, by way of non-limiting example, using a higher level of CBN compounds to create a more sedative experience. The individual terpenes may be formulated in such a way to mimic the aroma and flavors of naturally occurring cannabis.

In an embodiment, the added cannabinoid and/or terpene may be the same cannabinoid or terpene for which the target concentration in the extract-infused cannabis biomass is desired (i.e. the biomass cannabinoid or biomass terpene). In such embodiments, the additive cannabinoid or additive terpene is included in the calculation to arrive at the target concentration for that cannabinoid or terpene in the extract-infused cannabis biomass. In certain embodiments, the additive cannabinoid or additive terpene is added to the cannabis extract at a predefined amount, wherein the predefined amount may refer to an amount of the additive cannabinoid or additive terpene which provides, in addition to one or more of the at least one extract-derived cannabinoid, the at least one extract-derived terpene, the at least one plant-derived cannabinoid and the at least one plant-derived terpene, the target concentration of the homogeneous extract infused cannabis biomass or product. In certain embodiments, the at least one additive terpene may be provided at a weight ratio of the at least one additive terpene to the cannabis extract of no greater than 0.3, and preferably the weight ratio is between about 0.15 and about 0.25.

In certain embodiments, the method may comprise after the step of mixing, a step of screening (8) the homogeneous extract-infused cannabis biomass to improve homogeneity. In certain embodiments, the step of screening may comprise a physical screen. In certain embodiments, the step of screening the homogeneous extract-infused cannabis material may comprise visually screening to remove and/or homogenize a non-homogeneous extract-infused cannabis. In certain embodiments, the step of screening the homogeneous extract-infused cannabis material may comprise visually screening to remove an undesired material from the homogeneous extract-infused cannabis. In certain embodiments, the step of screening the homogeneous extract-infused cannabis material may comprise mechanically screening the extract-infused plant material or the homogeneous extract-infused biomass using a screen. In certain embodiments, the step of screening the homogeneous extract-infused cannabis material may comprise one or more of the aforementioned techniques or methods. In certain embodiments, the step of screening may comprise removing any undesirable material, such as plant material including, for example, stems. In certain embodiments, the step of screening may comprise a screen with a perforation size of about 4.8 mm.

In certain embodiments, the mixing may be repeated one or more times (13) to obtain the homogeneous extract-infused cannabis biomass. In certain embodiments, the mixing may be repeated one or times to obtain the homogeneous extract-infused cannabis biomass, wherein non-homogeneous extract-infused cannabis biomass may be screened then removed and/or remixed.

In certain embodiments, the extract-applied cannabis plant material may be frozen or cooled (9) prior to or during the mixing step to improve distribution of the cannabis extract throughout the plant material and/or to improve distribution of extract-infused plant material throughout the entirety of the mass of the plant material. Without being bound by any particular theory, warmed cannabis extract may exhibit difficulties in distribution due its stickiness. It has been observed (data not included) that by cooling or freezing the extract-applied cannabis plant, it is possible to improve distribution of the cannabis extract throughout the plant material.

In certain embodiments, the extract-applied plant material may be frozen or cooled at a temperature between about −30° C. to about −1° C., more particularly between about −30° C. to about −10° C., and more particularly still between about −20° C. to about −10° C. In an embodiment, the extract-applied plant material may be frozen or cooled to a temperature of about −1° C., about −2° C., about −3° C., about −4° C., about −5° C., about −6° C., about −7° C., about −8° C., about −9° C., about −10° C., about −11° C., about −12° C., about −13° C., about −14° C., about −15° C., about −16° C., about −17° C., about −18° C., about −19° C., about −20° C., or less, to produce a frozen extract-applied plant material, a homogeneous extract-infused biomass, or a frozen homogeneous extract-infused biomass, or any product thereof. The production of a frozen extract-applied plant material or a frozen homogeneous extract-infused biomass may provide advantages such as storage of the plant material or biomass, altering the material properties of the plant material and/or biomass such that downstream product formation steps including, but not limited to, forming a pre-rolled joint are more efficiently performed. The skilled person, in light of the teachings herein, would appreciate an extract-infused cannabis biomass prepared with greater amount of liquid extract may be more amenable to product formation when provided as a frozen homogeneous extract-infused biomass than those with a lower amount of liquid cannabis extract.

As will be appreciated from the disclosure herein, the methods herein advantageously provide a. Thus, in an embodiment, the present disclosure provides a homogeneous extract-infused biomass produced by the methods as described herein. The homogenous extract-infused cannabis biomass may be used to produce any number of products, including pre-rolled joints.

In an embodiment, the present disclosure provides a homogeneous extract-infused cannabis biomass comprising a target concentration of at least one cannabinoid, at least one terpene, or any combination thereof, wherein at least a portion of the target concentration of the at least one cannabinoid, at least one terpene, or combination thereof is from an exogenous source to an originating biomass of the extract-infused cannabis biomass, and wherein there is less than 15% variability in the target concentration of the at least one cannabinoid, at least one terpene, or combination thereof throughout a prepared batch or between alternates batches of the homogenous extract-infused cannabis biomass.

In an embodiment of the homogeneous extract-infused cannabis biomass of the present disclosure, the exogenous source is a cannabis extract. In an embodiment, the cannabis extract is a cannabis concentrate, a cannabis distillate, a cannabis oil, a cannabis isolate, a cannabis resin, or any combination thereof. In an embodiment, the cannabis extract is a cannabis concentrate, a cannabis distillate, or a combination thereof.

In an embodiment of the homogeneous extract-infused cannabis biomass of the present disclosure, the target concentration is of at least one biomass cannabinoid, and a target amount of the at least one biomass cannabinoid is provided by a cannabis extract having a predefined concentration of at least one extract-derived cannabinoid, a cannabis plant material having a predefined concentration of at least one plant-derived cannabinoid, an additive cannabinoid, or any combination thereof, in accordance with the present disclosure.

In an embodiment of the homogeneous extract-infused cannabis biomass of the present disclosure, the target concentration is of at least one biomass terpene, and a target amount of the at least one biomass terpene is provided by a cannabis extract having a predefined concentration of at least one extract-derived terpene, a cannabis plant material having a predefined concentration of at least one plant-derived terpene, an additive terpene, or any combination thereof, in accordance with the present disclosure.

In an embodiment of the homogeneous extract-infused cannabis biomass of the present disclosure, the target concentration is independently of at least one biomass cannabinoid and at least one biomass terpene, and a target amount of the at least one biomass cannabinoid and the at least one biomass terpene is provided by a cannabis extract having a predefined concentration of at least one extract-derived cannabinoid and/or terpene, a cannabis plant material having a predefined concentration of at least one plant-derived cannabinoid and/or terpene, an additive cannabinoid and/or terpene, or any combination thereof, in accordance with the present disclosure.

In an embodiment of the homogeneous extract-infused cannabis biomass of the present disclosure, there is between about 1% and about 15% variability in the target concentration of the at least one biomass cannabinoid, the at least one biomass terpene, or any combination thereof throughout the prepared batch, between the alternate batches, or both. In certain embodiments, there is about 14.5% variability, about 14% variability, about 13.5% variability, about 13% variability, about 12.5% variability, about 12% variability, about 11.5% variability, about 11% variability, about 10.5% variability, about 10% variability, about 9.5% variability, about 9% variability, about 8.5% variability, about 8% variability, about 7.5% variability, about 8% variability, about 7.5% variability, about 7% variability, about 6.5% variability, about 6% variability, about 5.5% variability, about 5% variability, about 4.5% variability, about 4.0% variability, about 3.5% variability, about 3.0% variability, about 2.5% variability, about 2% variability, about 1.5% variability, about 1% variability, about 0.5% variability in the target concentration of the at least one biomass cannabinoid, the at least one biomass terpene, or any combination thereof throughout the prepared batch, between the alternate batches, or both.

In certain embodiments, the homogeneous extract-infused cannabis biomass may be suitable for use in a pre-rolled joint. As used herein, “suitable for use” may comprise a pre-rolled joint that is substantially homogeneous throughout. In certain embodiments, suitable for use may comprise uniform colour throughout the homogeneous extract-infused biomass comprising the pre-rolled joint. In certain embodiments, “suitable for use” may comprise a uniform colour throughout the rolling paper or wrap used to form the pre-rolled joint, wherein there is no presence of oily residue on the rolling paper or wrap. In certain embodiments, suitable for use may comprise a pre-rolled joint, wherein if taken apart, the biomass of the extract-infused cannabis plant material is substantially free of sticky clumps. In certain embodiments, suitable for use may comprise a pre-rolled joint that is free of oily residue leaching through to the external surface of the wrap or paper. In certain embodiments, suitable for use may comprise a pre-rolled joint wherein if taken apart, the homogeneous extract infused cannabis biomass falls apart easily without external force.

In certain embodiments, there is provided a method of preparing a pre-rolled joint, the method comprising providing a homogeneous extract-infused biomass prepared according to the method described herein; freezing or cooling (10) the homogeneous extract-infused cannabis biomass (24) to produce a frozen extract-infused cannabis biomass (25); providing (11) the frozen extract-infused cannabis biomass (25) to a filling device and forming a pre-rolled smokable product comprising the frozen extract-infused cannabis product (26), wherein the filling device is configured to provide the pre-rolled smokable product; and tamping (12) the pre-rolled smokable product (26) to a desired packing density to produce the homogeneous extract-infused cannabis pre-rolled joint (27).

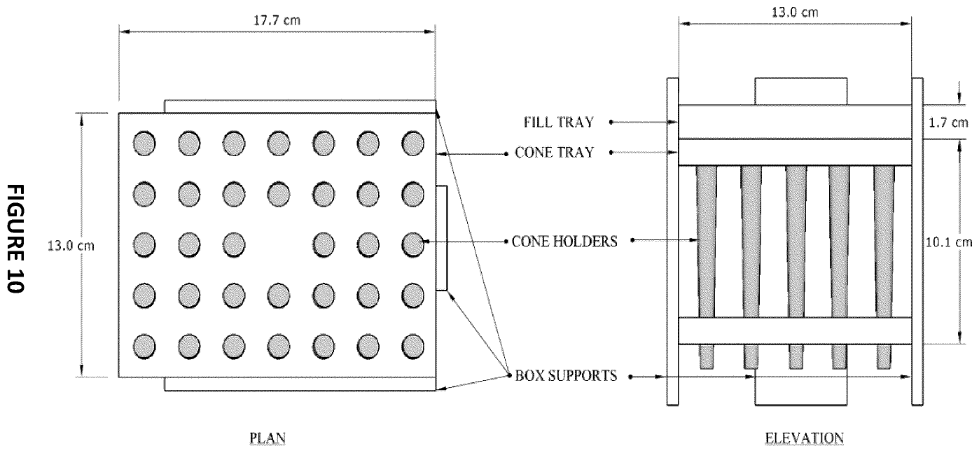

In certain embodiments, a filling device, such as that shown in FIG. 10, may be used to produce homogeneous extract-infused products, such as pre-rolled joints. In certain embodiments, the filling device may comprise a vibrator tray, a perforated plastic box comprising a cone tray and cone holder, wherein a cone may comprise a wrap or paper, and a fill tray. In certain embodiments, empty cones may be placed inside the cone tray. In certain embodiments, the vibratory tray may vibrate the homogeneous extract-infused biomass such that the homogeneous extract-infused biomass is provided to the empty cones in the cone tray.

In certain embodiments, the pre-rolled joints may be tampered to a desired packing density, and either folded or twisted to seal the pre-roll prior to packaging. The skilled person, in light of the teachings herein, would be able to select an appropriate method to tamp and seal the pre-rolled joint prior to packaging. In certain embodiments, the steps of filing, tampering and folding/twisting may be done using an automated machine equipped with appropriate multi-station technology.

### Exemplary Pre-Rolled Joints of the Present Disclosure

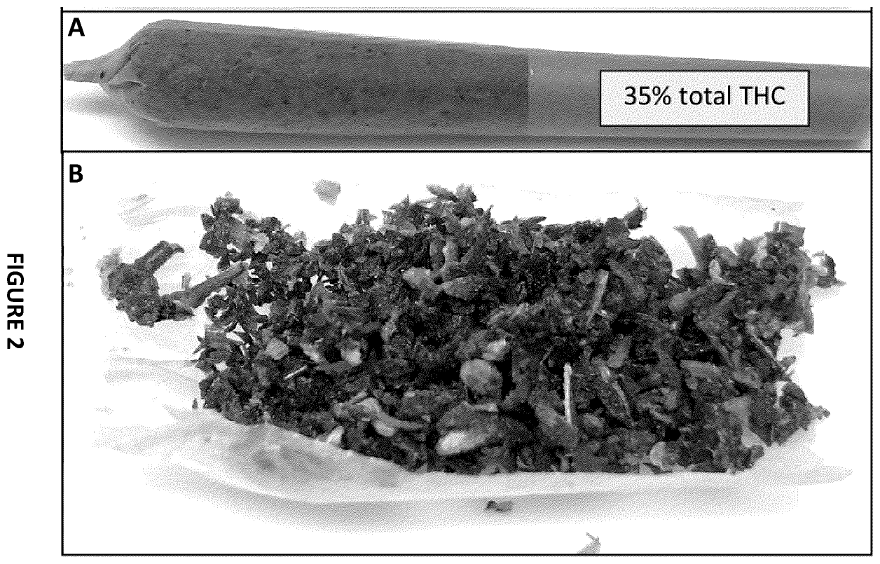

Examples of products as described herein are depicted in FIGS. 2, 3 and 4 (panels B & C).

In an embodiment, the present disclosure provides a pre-rolled joint. The pre-rolled is made with the homogenous extract-infused cannabis biomass of the present disclosure and/or prepared by the methods herein.

In certain embodiments, there is provided herein, a pre-rolled joint comprising a wrap or paper; a homogeneous extract-infused cannabis biomass (24) contained within the wrap or paper, the homogeneous extract-infused cannabis biomass comprising a target concentration of at least one cannabinoid, at least one terpene, or any combination thereof; and a filter, crutch, tip or mouthpiece disposed at a smoking end of the pre-rolled joint, wherein at least a portion of the target concentration of the at least one cannabinoid, at least one terpene, or combination thereof is from an exogenous source to an originating biomass of the extract-infused cannabis biomass, and wherein there is less than 15% variability in the target concentration of the at least one cannabinoid, at least one terpene, or combination thereof throughout the homogeneous extract-infused cannabis biomass contained within the pre-rolled joint.

The pre-rolled joint comprises a wrap or paper. A person skilled in the art would appreciate that paper or wrap may be rolled around a cannabis plant material or a homogeneous extract-infused cannabis biomass to provide a smokable product, which may be referred to as a “joint”. As used herein, a “pre-rolled” joint may be a joint provided, in which the joint was rolled by the manufacturer or provider, to the user, such that the user may not perform any steps to provide the “pre-rolled” joint. A person skilled in the art, in light of the teachings here, would be able to select an appropriate wrap or paper to produce a pre-rolled joint of the predefined cannabinoid and/or terpene amount. The paper or wrap, as would be known the person of skill, may provide a structure such that a smokable product, such as a pre-rolled joint, can be filled with a material, such as a homogeneous extract-infused cannabis biomass, to provide the product.

In certain embodiments, the pre-rolled joint may comprise an originating biomass. The “originating biomass” as used herein may refer to a cannabis plant material that is for infusion and/or mixing with a cannabis extract or is to be infused and/or mixed with a cannabis extract. The skilled person, in light of the teachings herein, would be able to select an appropriate originating biomass for a given predefined cannabinoid and/or terpene concentration. The skilled person may select the originating biomass in regard to properties such as, but not limited to, taste, terpene content, terpene amount, terpene concentration, ease of harvest, cannabinoid level, cannabinoid content, cannabinoid amount, concentrate absorption, concentrate retention, or any combination thereof.

In certain embodiments, the homogeneous extract-infused cannabis biomass may comprise at least one cannabinoid, at least one terpene, or any combination thereof from an exogenous source to an originating biomass of the extract-infused cannabis biomass. As used herein, the “exogenous source” to the originating biomass of the extract-infused cannabis biomass may comprise any source of the at least one cannabinoid, at least one terpene, or combination thereof that is not from the same biomass. In certain embodiments, not from the same biomass may comprise cannabis plant material and cannabis extract provided by the same cannabis strain or a different cannabis strain. In certain embodiments, the originating biomass is a cannabis plant material and the exogenous source is a cannabis extract.

In certain embodiments, the pre-rolled joint may comprise a filter, crutch, tip or mouthpiece disposed at a smoking end of the pre-rolled joint. The person of skill would appreciate that features such as the filter, crutch, tip and mouthpiece may provide one or more of the following: a means to prevent debris from entering the mouth of the user while consuming the product, a structural means to hold and consume the product, and a structural means to prevent burning the users fingers and/or mouth while consuming the product. The skilled person would be able to select an appropriate filter, crutch, tip or mouthpiece for an application as described herein.

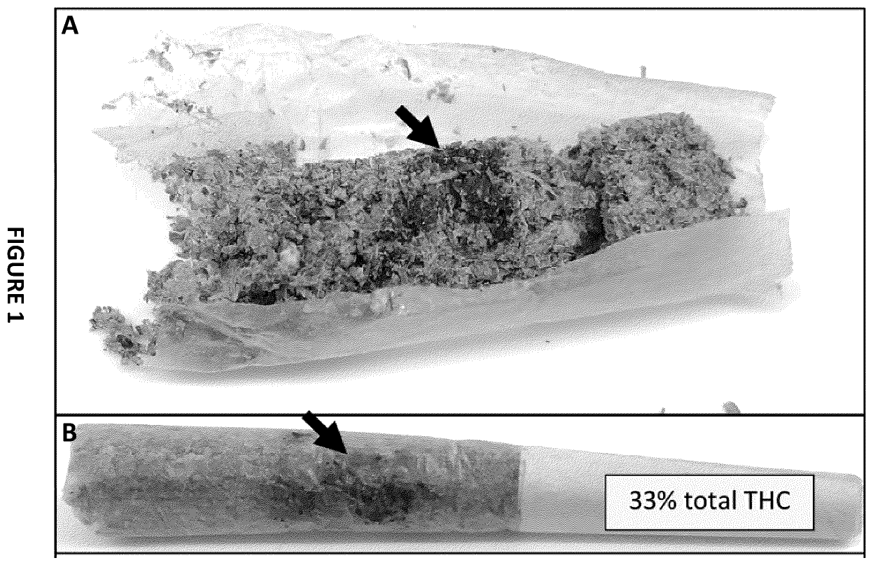

As shown in FIG. 1B, extract-infused cannabis pre-rolled products produced using conventional techniques may provide a product with heterogeneous or non-uniform distribution of the cannabis extract, as evidenced by distinct patches of dark coloured plant material, which a person of skill would appreciate may be due to insufficient homogenization. A person of skill would appreciate that non-uniform or heterogeneous distribution of the extract may alter the physical properties of the plant material due to oversaturation of the plant material with the extract. The extract-infused product of FIG. 1A produces a tightly packed cylinder of cannabis material which retains its shape when the pre-rolled joint is opened to expose the contents thereof. A person of skill in the art would appreciate that such tightly packed form may impair consumption of the smokable product, as this may impair air circulation and thus impair burning of the product when consumed. As such, the impaired burning may result in uneven burning of the product or an inability to burn the product or portions thereof.

In certain embodiments, the pre-rolled joint of the present disclosure, if taken apart, contains extract-infused cannabis biomass material that is substantially free of sticky clumps. In certain embodiments, the pre-rolled joint of the present disclosure, if taken apart, contains extract-infused cannabis biomass that falls apart easily without external force. In certain embodiments, it is contemplated that, during smoking of the pre-rolled joint of the present disclosure, the inhaled concentration, amount or potency of any one or more cannabinoids or terpenes exhibits less than 10% variability from start to finish, less than 7.5% variability from start to finish, less than 5% variability from start to finish, less than 4% variability from start to finish, between about 1% and about 10% variability from start to finish, or between about 3% and about 7% variability from start to finish of the pre-rolled joint.

Solvents, as would be known to the person of skilled, may be undesirable in a cannabis product including, but not limited to, a cannabis extract, a pre-rolled joint or a homogeneous extract-infused cannabis biomass. In certain embodiments described herein, the homogeneous extract-infused cannabis biomass or the homogeneous extract-infused cannabis biomass may be free of any residual solvent. In certain embodiments, the homogeneous extract-infused cannabis biomass may be free of any residual solvent such that the homogeneous extract-infused cannabis biomass and/or the pre-rolled joint is within the tolerance limits referred to in subsection 93(3) or 94(2) or section 101.1 of the Canadian Cannabis Regulations The person skilled in the art would appreciate that Class III solvents may be applied to the plant material. As would be known to the skilled person, Class Ill solvents may comprise Acetic acid, Heptane, Acetone, Isobutyl acetate, Anisole, Isopropyl acetate, 1-Butanol, Methyl acetate, 2-Butanol, 3-Methyl-1-butanol, Butyl acetate, Methylethylketone, tert-Butylmethyl ether, Methylisobutylketone, Cumene, 2-Methyl-I-propanol, Dimethyl sulfoxide, Pentane, Ethanol, 1-Pentanol, Ethyl acetate, 1-Propanol, Ethyl ether, 2-Propanol, Ethyl formate, Propyl acetate, or Formic Acid. In certain embodiments, the homogeneous extract-infused cannabis biomass may be free of Class III solvents. In certain embodiments, the pre-rolled joint may be free of ethanol.

The following examples are provided for illustrative purposes and are intended for the person of skill in the art. These examples are provided to demonstrate certain embodiments as described herein, and should not be seen as limiting in any way.

### Embodiments

In exemplary and non-limiting embodiments, the present disclosure relates to the following:

(1) In an embodiment, a method for preparing a homogeneous extract-infused cannabis biomass, the method comprising:
providing a cannabis extract having a predefined concentration of at least one extract-derived cannabinoid, at least one extract-derived terpene, or any combination thereof;
heating the cannabis extract at a temperature of up to 120° C. to provide a heated cannabis extract, wherein the heated cannabis extract is in a substantially liquid or flowable state;
providing a cannabis plant material having a predefined concentration of at least one plant-derived cannabinoid, at least one plant-derived terpene, or any combination thereof, wherein the cannabis plant material is a milled or comminuted cannabis plant material;
applying the heated cannabis extract to the cannabis plant material using a predefined ratio of the cannabis plant material to the heated cannabis extract to provide an extract-applied plant material, wherein the predefined ratio provides a target amount of at least one biomass cannabinoid, at least one biomass terpene, or any combination thereof within the extract-applied plant material; and
mixing the extract-applied plant material to provide a homogeneous extract-infused cannabis biomass, wherein there is less than a 15% variability in a target concentration of the at least one biomass cannabinoid, the at least one biomass terpene, or any combination thereof throughout a prepared batch and/or between alternate batches of the homogeneous extract-infused cannabis biomass.

(2) In an embodiment, the method of (1), wherein there is less than a 10% variability in the target concentration of the at least one biomass cannabinoid, the at least one biomass terpene, or any combination thereof throughout the prepared batch, between the alternate batches, or both.

(3) In an embodiment, the method of (1) or (2), wherein there is less than a 7.5% variability in the target concentration of the at least one biomass cannabinoid, the at least one biomass terpene, or any combination thereof throughout the prepared batch, between the alternate batches, or both.

(4) In an embodiment, the method of any one of (1) to (3), wherein there is less than a 5% variability in the target concentration of the at least one biomass cannabinoid, the at least one biomass terpene, or any combination thereof throughout the prepared batch, between the alternate batches, or both.

(5) In an embodiment, the method of any one of (1) to (4), wherein there is less than a 4% variability in the target concentration of the at least one biomass cannabinoid, the at least one biomass terpene, or any combination thereof throughout the prepared batch, between the alternate batches, or both.

(6) In an embodiment, the method of any one of (1) to (5), wherein there is between about 0.5% to about 10% variability in the target concentration of the at least one biomass cannabinoid, the at least one biomass terpene, or any combination thereof throughout the prepared batch, between the alternate batches, or both.

(7) In an embodiment, the method of any one of (1) to (5), wherein there is between about 3% to about 7% variability in the target concentration of the at least one biomass cannabinoid, the at least one biomass terpene, or any combination thereof throughout the prepared batch, between the alternate batches, or both.

(8) In an embodiment, the method of any one of (1) to (5), wherein the homogeneous extract-infused cannabis biomass has substantially no variability in the target concentration of the at least one biomass cannabinoid, the at least one biomass terpene, or any combination thereof throughout the prepared batch, between the alternate batches, or both.

(9) In an embodiment, the method of any one of (1) to (8), wherein the target amount is of the at least one biomass cannabinoid, the at least one biomass terpene, or both, and:
the target amount of the at least one biomass cannabinoid is obtained by the at least one extract-derived cannabinoid, the at least one plant-derived cannabinoid, or both; and
the target amount of the at least one biomass terpene is obtained by the at least one extract-derived terpene, the at least one plant-derived terpene, or both.

(10) In an embodiment, the method of any one of (1) to (9), wherein the target amount is of the at least one biomass cannabinoid, and the target amount is obtained by the at least one extract-derived cannabinoid or the at least one plant-derived cannabinoid.

(11) In an embodiment, the method of any one of (1) to (9), wherein the target amount is of the at least one biomass cannabinoid, and the target amount is obtained by a combination of the at least one extract-derived cannabinoid and the at least one plant-derived cannabinoid.

(12) In an embodiment, the method of any one of (1) to (9), wherein the target amount is of the at least one biomass terpene, and the target amount is obtained by the at least one extract-derived terpene or the at least one plant-derived terpene.

(13) In an embodiment, the method of any one of (1) to (9), wherein the target amount is of the at least one biomass terpene, and the target amount is obtained by a combination of the at least one extract-derived terpene and the at least one plant-derived terpene.

(14) In an embodiment, the method of any one of (1) to (9), wherein the target amount is of the at least one biomass cannabinoid and the at least one biomass terpene, and the target amount is obtained by a combination of (i) the at least one extract-derived cannabinoid and the at least one extract-derived terpene; and (ii) the at least one plant-derived cannabinoid and the at least one plant-derived terpene.

(15) In an embodiment, the method of any one of (1) to (8), further comprising a step of providing at least one additive to the cannabis extract prior to the applying of the heated cannabis extract to the cannabis plant material.

(16) In an embodiment, the method of (15), wherein the at least one additive comprises at least one additive terpene, at least one additive cannabinoid, or any combination thereof, wherein the at least one additive is added to the cannabis extract at a predefined amount.

(17) In an embodiment, the method of (16), which comprises providing the at least one additive terpene and wherein the at least one additive terpene is provided at a weight ratio of the at least one additive terpene to the cannabis extract of no greater than 0.3, and preferably the weight ratio is between about 0.15 and about 0.25.

(18) In an embodiment, the method of any one of (15) to (17), wherein the target amount is of the at least one biomass cannabinoid, the at least one biomass terpene, or both, and:
the target amount of the at least one biomass cannabinoid is obtained from one or more of the at least one extract-derived cannabinoid, the at least one plant-derived cannabinoid, and the at least one additive-derived cannabinoid; and
the target amount of the at least one biomass terpene is obtained from one or more of the at least one extract-derived terpene, the at least one plant-derived terpene, and the at least one additive-derived terpene.

(19) In an embodiment, the method of any one of (15) to (18), wherein the target amount is of the at least one biomass cannabinoid, and the target amount is obtained by a combination of the at least one additive-derived cannabinoid and the at least one extract-derived cannabinoid or the at least one plant-derived cannabinoid.

(20) In an embodiment, the method of any one of (15) to (18), wherein the target amount is of the at least one biomass cannabinoid, and the target amount is obtained by a combination of the at least one extract-derived cannabinoid, the at least one plant-derived cannabinoid, and the at least one additive-derived cannabinoid.

(21) In an embodiment, the method of any one of (15) to (18), wherein the target amount is of the at least one biomass terpene, and the target amount is obtained by a combination of the at least one additive-derived terpene and the at least one extract-derived terpene or the at least one plant-derived terpene.

(22) In an embodiment, the method of any one of (15) to (18), wherein the target amount is of the at least one biomass terpene, and the target amount is obtained by a combination of the at least one extract-derived terpene, the at least one plant-derived terpene, and the at least one additive-derived terpene.

(23) In an embodiment, the method of any one of (15) to (18), wherein the target amount is of the at least one biomass cannabinoid and the at least biomass terpene, and the target amount is obtained by a combination of (i) the at least one additive-derived cannabinoid and the at least one extract-derived cannabinoid or the at least one plant-derived cannabinoid, and (ii) the at least one additive-derived terpene and the at least one extract-derived terpene or the at least one plant-derived terpene.

(24) In an embodiment, the method of any one of (15) to (18), wherein the target amount is of the at least one biomass cannabinoid and the at least one biomass terpene, and the target amount is obtained by a combination of (i) the at least one extract-derived cannabinoid, the at least one plant-derived cannabinoid, and the at least one additive-derived cannabinoid, and (ii) the at least one extract-derived terpene, the at least one plant-derived terpene, and the at least one additive-derived terpene.

(25) In an embodiment, the method of any one of (1) to (24), wherein the mixing is repeated one or more times to obtain the homogeneous extract-infused cannabis biomass.

(26) In an embodiment, the method of any one of (1) to (25), wherein the step of mixing the extract-applied plant material comprises a step of freezing or cooling the extract-applied plant material.

(27) In an embodiment, the method of (26), which comprises freezing the extract-applied plant material at a temperature between about −30° C. to about −10° C. to produce a frozen extract-applied plant material.

(28) In an embodiment, the method of (27), further comprising mixing the frozen extract-applied plant material to provide the homogeneous extract-infused cannabis biomass.

(29) In an embodiment, the method of any one of (1) to (28), further comprising, after the mixing, a step of screening the homogeneous extract-infused cannabis biomass to improve homogeneity.

(30) In an embodiment, the method of any one of (1) to (29), wherein the applying is by pouring, sprinkling or showering the heated cannabis extract onto the cannabis plant material.

(31) In an embodiment, the method of any one of (1) to (30), wherein the mixing is by rotational mixing, blending, stirring, beating, kneading, or any combination thereof of the extract-applied plant material.

(32) In an embodiment, the method of any one of (1) to (31), wherein the applying and the mixing steps are performed simultaneously.

(33) In an embodiment, the method of any one of (1) to (32), wherein the mixing is performed in a mixer comprising a flat beater attachment and the flat beater attachment mixes the cannabis plant material and the heated cannabis extract.

(34) In an embodiment, the method of (33), wherein the flat beater attachment mixes the cannabis plant material and the heated cannabis extract at between about 80 rpm and about 120 rpm.

(35) In an embodiment, the method of any one of (1) to (34), wherein in the step of heating the cannabis extract, the temperature is between about 60° C. and about 120° C.

(36) In an embodiment, the method of any one of (1) to (35), wherein in the step of heating the cannabis extract, the temperature is between about 60° C. and about 100° C.

(37) In an embodiment, the method of any one of (1) to (36), wherein in the step of heating the cannabis extract, the temperature is about 80° C.

(38) In an embodiment, the method of any one of (1) to (37), wherein the cannabis extract contains minimal or no residual solvent used in an extraction process.

(39) In an embodiment, the method of any one of (1) to (38), wherein the cannabis extract is a cannabis concentrate, a cannabis distillate, a cannabis oil, a cannabis isolate, a cannabis resin, or any combination thereof.

(40) In an embodiment, the method of any one of (1) to (39), wherein the cannabis extract is a cannabis distillate.

(41) In an embodiment, the method of any one of (1) to (39), wherein the cannabis extract is a cannabis concentrate.

(42) In an embodiment, the method of any one of (1) to (41), wherein the target amount of the biomass cannabinoid is of tetrahydrocannabiorcol (THCC), Δ9-tetrahydrocannabinol (Δ9-THC), Δ8-tetrahydrocannabinol (Δ8-THC), cannabidiol (CBD), cannabigerol (CBG), cannabichromene (CBC), cannabinol (CBN), cannabicyclol (CBL), cannabielsoin (CBE), cannabivarin (CBV), cannabifuran (CBF), cannabitran (CBT), cannabiripsol (CBR), any acidic forms thereof, or any combination thereof.

(43) In an embodiment, the method of (42), wherein the target amount of the biomass cannabinoid is of Δ9-THC, CBD, CBG, any neutral versions thereof, or any combination thereof.

(44) In an embodiment, the method of any one of (1) to (43), wherein the target amount of the biomass terpene is of bisabolol, borneol, camphene, carene, caryophyllene, eucalyptol, farnesene, geraniol, humulene, limonene, linalool, myrcene, nerolido, farnesene, pinene, terpineol, valencene, hemiterpenoids, monoterpenoid, sesquiterpenoid, diterpenoid, trans-caryophyllene, sesterterpenoid, triterpenoid, tetraterpenoid, polyterpenoid, citral, menthol, camphor, salvinorin A, ginkgolide or any combination thereof.

(45) In an embodiment, the method of (44), wherein the target amount of the biomass terpene is of caryophyllene, farnesene, linalool, or any combination thereof.

(46) In an embodiment, the method of any one of (1) to (45), wherein the cannabis plant material is whole plant, buds, flower, flower portions, bark, rhizomes, fruit, or any combination thereof.

(47) In an embodiment, the method of (46), wherein the cannabis plant material is dried flower.

(48) In an embodiment, the method of any one of (1) to (47), wherein the milled or comminuted cannabis plant material is of a particle size of less than or equal to 5.0 mm.

(49) In an embodiment, the method of (48), wherein the particle size is between about 100 μm and about 3.5 mm.

(50) In an embodiment, the method of any one of (1) to (49), wherein a mass of the cannabis extract; the predefined concentration of the at least one extract-derived cannabinoid; the predefined concentration of the at least one extract-derived terpene; a mass of the cannabis plant material; the predefined concentration of the at least one plant-derived cannabinoid; the predefined concentration of the at least one plant-derived terpene; or any combination thereof is calculated using:

$$E = \frac{[ A \times B ] + [ C \times D ]}{[ A + C ]}$$

wherein A is the mass of the cannabis plant material, B is the concentration of the at least one plant-derived cannabinoid or the concentration of the at least one cannabis plant-derived terpene, C is the mass of the cannabis extract, D is the concentration of the at least one extract-derived cannabinoid or the concentration of the at least one extract-derived terpene, and E is the target concentration of the at least one biomass cannabinoid or the at least one biomass terpene.

(51) In an embodiment, the method of any one of (1) to (50), wherein a length of time for the heating of the cannabis extract is calculated using:

$$The length of time for heating of the cannabis extract ( s ) = \frac{m \times cp \times Δ T}{P}$$

wherein m is a mass of the cannabis extract, cp is a specific heat capacity of the cannabis extract in J/kg° C., ΔT is a temperature change (° C.) of the cannabis extract; and P is a rate of energy (W) transfer to the cannabis extract.

(52) In an embodiment, the method of any one of (1) to (51), wherein the homogeneous extract-infused cannabis biomass comprises at least 55% [w/w], at least 60% [w/w], at least 70% [w/w], or at least 75% [w/w], of the cannabis plant material.

(53) In an embodiment, the method of any one of (1) to (52), wherein the variability in the target concentration of the at least one biomass cannabinoid, the at least one biomass terpene, or any combination thereof is throughout the prepared batch.

(54) In an embodiment, the method of any one of (1) to (52), wherein the variability in the target concentration of the at least one biomass cannabinoid, the at least one biomass terpene, or any combination thereof is between alternate batches.

(55) In an embodiment, the method of any one of (1) to (52), wherein the variability in the target concentration of the at least one biomass cannabinoid, the at least one biomass terpene, or any combination thereof is throughout the prepared batch and between alternate batches.

(56) In an embodiment, the method of any one of (1) to (55), wherein the homogeneous extract-infused cannabis biomass is suitable for use in a pre-rolled joint.

(57) In an embodiment, the method of (56), further comprising a step of providing the homogeneous extract-infused cannabis biomass to a filling device, wherein the filling device is configured to provide the pre-rolled joint.

(58) In an embodiment, a homogeneous extract-infused cannabis product prepared from the homogeneous extract-infused cannabis biomass according to the method of any one of (1) to (55).

(59) In an embodiment, the homogeneous extract-infused cannabis product of (58), which is a pre-rolled joint.

(60) In an embodiment, the homogeneous extract-infused cannabis product of (59), wherein the homogeneous extract-infused cannabis biomass is frozen or cooled in order to produce the pre-rolled joint.

(61) In an embodiment, the homogeneous extract-infused cannabis product of (60), wherein the homogeneous extract-infused cannabis biomass is frozen or cooled at between about −30° C. to about −10° C.

(62) In an embodiment, a homogeneous extract-infused cannabis biomass produced by the method of any one of (1) to (55).

(63) In an embodiment, the homogeneous extract-infused cannabis biomass of (62), which is for use in a pre-rolled joint.

(64) In an embodiment, the homogeneous extract-infused cannabis biomass of (63), comprising providing the homogeneous extract-infused cannabis biomass to a filling device, wherein the filling device is configured to provide the pre-rolled joint.

(65) In an embodiment, the homogeneous extract-infused cannabis biomass of (63) or (64), wherein the extract-infused cannabis biomass is frozen or cooled in order to produce the pre-rolled joint.

(66) In an embodiment, the homogeneous extract-infused cannabis biomass of (65), wherein the extract-infused cannabis biomass is frozen or cooled at between about −30° C. to about −10° C.

(67) In an embodiment, a pre-rolled joint comprising:
a wrap or paper;
a homogeneous extract-infused cannabis biomass contained within the wrap or paper, the homogeneous extract-infused cannabis biomass comprising a target concentration of at least one cannabinoid, at least one terpene, or any combination thereof; and
a filter, crutch, tip or mouthpiece disposed at a smoking end of the pre-rolled joint,
wherein at least a portion of the target concentration of the at least one cannabinoid, at least one terpene, or combination thereof is from an exogenous source to an originating biomass of the extract-infused cannabis biomass, and wherein there is less than 15% variability in the target concentration of the at least one cannabinoid, at least one terpene, or combination thereof throughout the homogeneous extract-infused cannabis biomass contained within the pre-rolled joint.

(68) In an embodiment, the pre-rolled joint of (67), wherein there is less than 10% variability in the target concentration of the at least one cannabinoid, at least one terpene, or combination thereof throughout the homogeneous extract-infused cannabis biomass contained within the pre-rolled joint.

(69) In an embodiment, the pre-rolled joint of (67), wherein there is less than 7.5% variability in the target concentration of the at least one cannabinoid, at least one terpene, or combination thereof throughout the homogeneous extract-infused cannabis biomass contained within the pre-rolled joint.

(70) In an embodiment, the pre-rolled joint of (67), wherein there is less than 5% variability in the target concentration of the at least one cannabinoid, at least one terpene, or combination thereof throughout the homogeneous extract-infused cannabis biomass contained within the pre-rolled joint.

(71) In an embodiment, the pre-rolled joint of (67), wherein there is less than 4% variability in the target concentration of the at least one cannabinoid, at least one terpene, or combination thereof throughout the homogeneous extract-infused cannabis biomass contained within the pre-rolled joint.

(72) In an embodiment, the pre-rolled joint of (67), wherein there is between about 1% to about 10% variability in the target concentration of the at least one cannabinoid, at least one terpene, or combination thereof throughout the homogeneous extract-infused cannabis biomass contained within the pre-rolled joint.

(73) In an embodiment, the pre-rolled joint of (67), wherein there is between about 3% to about 7% variability in the target concentration of the at least one cannabinoid, at least one terpene, or combination thereof throughout the homogeneous extract-infused cannabis biomass contained within the pre-rolled joint.

(74) In an embodiment, the pre-rolled joint of (67), wherein the homogeneous extract-infused cannabis biomass has substantially no variability in concentration of the at least one cannabinoid, at least one terpene, or combination thereof throughout the homogeneous extract-infused cannabis biomass contained within the pre-rolled joint.

(75) In an embodiment, the pre-rolled joint of any one of (67) to (74), wherein the homogeneous extract-infused cannabis biomass is as defined in any one of (1) to (55).

(76) In an embodiment, the pre-rolled joint of any one of (67) to (75), wherein the homogeneous extract-infused cannabis is prepared according to the method of any one of 1 to 55.

(77) In an embodiment, the pre-rolled joint of any one of (67) to (76), which is free of any residual solvent.

(78) In an embodiment, the pre-rolled joint of any one of (67) to (77), which is free of any Class 3 solvent.

(79) In an embodiment, the pre-rolled joint of any one of (67) to (78), which is free of ethanol.

(80) In an embodiment, the pre-rolled joint of any one of (67) to (79), which exhibits a smooth even burn.

(81) In an embodiment, the pre-rolled joint of any one of (67) to (80), wherein during smoking the inhaled concentration or potency of any one or more cannabinoids or terpenes exhibits less than 10% variability from start to finish, exhibits less than 7.5% variability from start to finish, exhibits less than 5% variability from start to finish, exhibits less than 4% variability from start to finish, exhibits between about 1% and about 10% variability from start to finish, or exhibits between about 3% and about 7% variability from start to finish.

(82) In an embodiment, the pre-rolled joint of any one of (70) to (87), which further comprises an additive.

(83) In an embodiment, the pre-rolled joint of (82), wherein the additive comprises at least one additive terpene, at least one additive cannabinoid, or any combination thereof, wherein the additive is provided at a predefined additive amount to provide the target concentration of the at least one cannabinoid, at least one terpene, or combination thereof.

(84) In an embodiment, the pre-rolled joint of any one of (67) to (83), which is free of oily residue leaching through to the external surface of the wrap or paper.

(85) In an embodiment, the pre-rolled joint of any one of (67) to (84), wherein if taken apart, the biomass of the extract-infused cannabis plant material is substantially free of sticky clumps.

(86) In an embodiment, the pre-rolled joint of any one of (70) to (91), wherein if taken apart, the homogeneous extract-infused cannabis biomass falls apart easily without external force.

(87) In an embodiment, a homogeneous extract-infused cannabis biomass comprising a predefined amount of at least one cannabinoid, at least one terpene, or any combination thereof, wherein at least a portion of the predefined amount of the at least one cannabinoid, the at least one terpene, or combination thereof is from an exogenous source to an originating cannabis plant material, and wherein there is less than 15% variability in concentration the at least one cannabinoid, at least one terpene, or combination thereof across any given mass of the homogeneous extract-infused cannabis biomass.

(88) In an embodiment, the homogeneous extract-infused cannabis biomass of (87), wherein the exogenous source is a cannabis extract, for example a cannabis distillate.

(89) In an embodiment, a method of preparing a pre-rolled joint, the method comprising:
providing the homogeneous extract-infused biomass prepared according to the method of any one of (1) to (55);
freezing or cooling the extract-infused cannabis biomass to produce a frozen extract-infused cannabis biomass;
providing the frozen extract-infused cannabis biomass to a filling device and forming a pre-rolled smokable product comprising the frozen extract-infused cannabis product, wherein the filling device is configured to provide the pre-rolled smokable product; and
tamping the pre-rolled smokable product to a desired packing density to produce the homogeneous extract-infused cannabis pre-rolled joint.

### EXAMPLES

### Example 1: THC Content of Exemplary Homogeneous Extract-Infused Cannabis Biomass

A total of 10 samples were randomly collected from two separate batches of homogeneous extract-infused cannabis biomass, herein referred to as ANC-186 and ANC-187 and sent to a third-party testing lab for testing.

In lot ANC-186, dried cannabis flower of the strain “Afghani Drifter” with a 21.91% total THC concentration was milled to a particle size of ≤3.2 mm. A cannabis concentrate with a 89.10% total THC concentration was heated for 30 minutes at 80° C. 55,760 grams of the milled cannabis was mixed with 19,600 grams of the cannabis concentrate providing a ratio of 70:30 and a total THC label claim of 40.00%. Mixing was performed by providing the milled cannabis plant material and the heated cannabis concentrate in a stainless-steel vessel to a mixer and mixing at 108 RPM in batches of 1,100 grams, until the entire lot was mixed to produce the homogeneous extract-infused cannabis biomass.

In lot ANC-187, dried cannabis flower of strain “Afghani Drifter” with a 21.94% total THC concentration was milled to a particle size of ≤3.2 mm. A cannabis concentrate with a 89.10% total THC concentration was heated for 30 minutes at 80° C. 55,040 grams of the milled cannabis was mixed with 19,500 grams of a cannabis concentrate providing a ratio of 70:30 and a total THC label claim of 40.00%. Mixing was performed by providing the milled cannabis and the heated cannabis concentrate in a stainless-steel vessel to a mixer and mixing at 108 RPM in batches of 1,100 grams, until the entire lot was mixed to provide the homogeneous extract-infused cannabis biomass.

TABLE 1
D9-THC, THCA-A, and total THC from 5 samples randomly collected
from pre-roll joints of lot ANC-186 and lot ANC-187.

| | | | |
|---|---|---|---|
| Sample | D9-THC (mg/g) | THCA (mg/g) | Total THC¹ (mg/g) |

LOT ANC-186

| | | | |
|---|---|---|---|
| 1 | 194.28 | 252.66 | 415.86 |
| 2 | 204.28 | 256.13 | 428.91 |
| 3 | 190.33 | 258.74 | 417.24 |
| 4 | 194.82 | 238.53 | 404.01 |
| 5 | 187.36 | 244.83 | 402.08 |
| Mean | 194.21 | 250.18 | 413.62 |
| SD | 5.72 | 7.47 | 9.77 |
| RSD | 2.95 | 2.99 | 2.36 |

Lot ANC-187

| | | | |
|---|---|---|---|
| 1 | 219.90 | 224.05 | 416.39 |
| 2 | 212.68 | 221.19 | 406.66 |
| 3 | 222.83 | 234.26 | 428.28 |
| 4 | 214.75 | 251.50 | 435.32 |
| 5 | 209.03 | 225.99 | 407.22 |
| Mean | 215.84 | 231.40 | 418.77 |
| SD | 4.96 | 10.95 | 11.40 |
| RSD | 2.30 | 4.73 | 2.72 |

¹Total THC = [(THCA × 0.877) + D9-THC]
0.877 is a conversion factor for THCA (358.48 g/mol) into THC (314.45 g/mol).
D9-THC: Delta-9-Tetrahydrocannabinol; THCA-A: Delta-9-Tetrahydrocannabinolic acid A; SD: Standard Deviation; RSD: Relative Standard Deviation.

Pre-rolls from lot ANC-186 had an average total THC amount of 413.62±9.77 mg/g, with an RSD of 2.4%. Similarly, pre-rolls from lot ANC-187 had an average total THC of 418.77±11.40 mg/g, with an RSD of 2.7%.

### Example 2: THC and Terpene Concentration of an Exemplary Homogeneous Extract-Infused Biomass

A total of 6 samples were randomly collected from lot ANC-492 and sent to a third-party testing lab for testing.

For lot ANC-492, dried cannabis flower of strain “GMO” with a 20.94% total THC concentration and dried cannabis flower of strain “Atomic Sour Grapefruit” with a 25.29% total THC concentration were milled to a particle size of ≤3.2 mm. A THC-containing cannabis distillate with a 91.33% total THC concentration was heated for 60 minutes at 120° C. Terpenes were added to the heated THC-containing distillate at a ratio of 11:89 to form a cannabis concentrate with 65.53% total THC concentration. A total of 18,316 grams of milled cannabis plant material (9,158 grams of the GMO flower and 9, 158 grams of the Atomic Sour Grapefruit flower) was mixed with 7,850 grams of the cannabis concentrate at a ratio of 70:30 to a total THC label claim of 35.00%. Mixing was performed by providing the milled cannabis plant material and the heated cannabis concentrate in a stainless-steel vessel to a mixer and mixing at 108 RPM in batches of 1,100 grams, until the entire lot was mixed to provide the homogeneous extract-infused cannabis biomass.

TABLE 2
Cannabinoid and terpene level from randomly collected
samples of infused pre-roll lot ANC-492.

| Cannabinoid Profile | | | | Terpene Profile | | | |
|---|---|---|---|---|---|---|---|
| | | | Total | | | Trans- | Total |
| | D9-THC | THCA | THC¹ | Linalool | Farnesene | Caryophyllene | Terpenes |
| Sample | (mg/g) | (mg/g) | (mg/g) | (wt %) | (wt %) | (wt %) | (wt %) |
| 1 | 237.84 | 159.73 | 377.92 | 0.24100 | 0.22100 | 0.16200 | 1.19800 |
| 2 | 246.23 | 162.15 | 388.44 | 0.23100 | 0.20900 | 0.16000 | 1.11600 |
| 3 | 242.90 | 168.07 | 390.29 | 0.22500 | 0.22200 | 0.15500 | 1.13300 |
| 4 | 254.03 | 155.44 | 390.36 | 0.24200 | 0.21900 | 0.15900 | 1.19200 |
| 5 | 237.41 | 150.01 | 368.97 | 0.21600 | 0.21500 | 0.15000 | 1.07900 |
| 6 | 235.98 | 160.21 | 376.48 | 0.22600 | 0.22300 | 0.15900 | 1.15500 |
| Mean | 242.40 | 159.27 | 382.08 | 0.23017 | 0.21817 | 0.15750 | 1.14550 |
| SD | 6.29 | 5.58 | 8.13 | 0.00915 | 0.00484 | 0.00395 | 0.04175 |
| RSD | 2.59 | 3.50 | 2.13 | 3.97735 | 2.22069 | 2.50640 | 3.64454 |

¹Total THC = [(THCA × 0.877) + D9-THC]
0.877 is a conversion factor for THCA (358.48 g/mol) into THC (314.45 g/mol).
D9-THC: Delta-9-Tetrahydrocannabinol; THCA-A: Delta-9-Tetrahydrocannabinolic acid A; SD: Standard Deviation; RSD: Relative Standard Deviation.

The randomly collected samples from extract infused pre-rolls of lot ANC-492 had an average total THC amount of 382.08±8.13 mg/g, and the terpene profile of the extract infused pre-rolls of lot ANC-492 showed uniform distribution of Linalool, Farnesene, and Trans-Caryophyllene throughout.

### Example 3: THC, Terpene and Minor Cannabinoids Concentration of an Exemplary an Homogeneous Extract-Infused Biomass

A total of 6 samples were randomly collected from lot ANC-539 and sent to a third-party testing lab for testing.

In lot ANC-539, dried cannabis flower of strain “Atomic Sour Grapefruit” with a 20.91% total THC concentration and 0.54% total CBG concentration was milled to a particle size of ≤3.2 mm. THC-containing cannabis distillate with a 91.33% total THC concentration and a 2.92% total CBG concentration was heated for 60 minutes at 120° C. Terpenes were added to the heated THC-containing distillate at a ratio of 16:84 to form a cannabis concentrate with 76.78% total THC concentration and 2.45% total CBG concentration. 2,040 grams of the milled cannabis was mixed with 2,480 grams of infused mill flower with a 35.62% total THC concentration and a 10.66% total CBG concentration, and 452 grams of the cannabis concentrate to a total THC label claim of 32.00% and a total CBG label claim of 5.00%. Mixing was performed by providing the milled cannabis plant material, infused mill biomass and the heated cannabis concentrate in a stainless-steel vessel to a mixer and mixing at 108 RPM in batches of 1,100 grams, until the entire lot was mixed to provide the homogeneous extract-infused cannabis biomass.

TABLE 3
Cannabinoid and terpene profiles from randomly collected samples of extract infused pre-rolls from lot ANC-539

| | Cannabinoid Profile | | | | | | Terpene Profile | | | |
|---|---|---|---|---|---|---|---|---|---|---|
| | | | Total | | | Total | | | Trans- | Total |
| | D9-THC | THCA | THC¹ | CBG | CBGA | CBG² | Linalool | Farnesene | Caryophyllene | Terpenes |
| Sample | (mg/g) | (mg/g) | (mg/g) | (mg/g) | (mg/g) | (mg/g) | (wt %) | (wt %) | (wt %) | (wt %) |
| 1 | 177.59 | 152.60 | 311.42 | 45.43 | 3.64 | 48.63 | 0.30500 | 0.29800 | 0.10700 | 0.99800 |
| 2 | 172.30 | 155.09 | 308.31 | 42.91 | 3.68 | 46.14 | 0.31100 | 0.29300 | 0.11000 | 1.00200 |
| 3 | 193.83 | 141.26 | 317.71 | 47.96 | 3.60 | 51.12 | 0.34700 | 0.32300 | 0.12300 | 1.11600 |
| 4 | 190.10 | 143.83 | 316.23 | 47.98 | 3.59 | 51.13 | 0.35200 | 0.34200 | 0.11700 | 1.14400 |
| 5 | 185.00 | 146.48 | 313.47 | 46.45 | 3.60 | 49.61 | 0.33200 | 0.32600 | 0.10600 | 1.07600 |
| 6 | 210.96 | 137.37 | 331.43 | 55.57 | 3.64 | 58.77 | 0.37400 | 0.33600 | 0.11600 | 1.15200 |
| Mean | 188.29 | 146.10 | 316.43 | 47.72 | 3.63 | 50.90 | 0.33683 | 0.31967 | 0.11317 | 1.08133 |
| SD | 12.44 | 6.16 | 7.38 | 3.91 | 0.03 | 3.90 | 0.02387 | 0.01825 | 0.00604 | 0.06244 |
| RSD | 6.61 | 4.22 | 2.33 | 8.19 | 0.84 | 7.67 | 7.08677 | 5.70758 | 5.33657 | 5.77396 |

¹Total THC = [(THCA × 0.877) + D9-THC]
²Total CBG = [(CBGA × 0.878) + CBG]
0.877 is a conversion factor for THCA (358.48 g/mol) into THC (314.45 g/mol)
0.878 is a conversion factor for CBGA (360.49 g/mol) into CBG (316.48 g/mol).
D9-THC: Delta-9-Tetrahydrocannabinol; THCA-A: Delta-9-Tetrahydrocannabinolic acid A; CBG: Cannabigerol; CBGA: Cannabigerolic acid; SD: Standard Deviation; RSD: Relative Standard Deviation.

The extract infused pre-rolls from lot ANC-539 had an average total THC amount of 316.43±7.38 mg/g and an average total CBG content of 50.90±3.90 mg/g. Furthermore, the terpene profile of randomly collected samples from the extract infused pre-rolls of lot ANC-539 showed uniform distribution of Linalool, Farnesene, and Trans-Caryophyllene throughout.

### Example 4: Exemplary Products B and C Produced Using Methods Defined Herein

Four pre-rolled joints (Products A-D) containing extract-infused cannabis plant material are depicted in FIGS. 1-4 were analyzed for visual homogeneity.

### Product A

Product A is an extract-infused pre-rolled joint produced using a conventional method comprising brown paper, a yellow filter, and 33% total THC. The extract used for preparing the pre-rolled joint of Product A was a cannabis distillate. When the internal plant material of Product A is exposed/removed, the rolled plant material retains its cylinder shape (see FIG. 1, panel A). The extract-infused cannabis plant material of Product A exhibits heterogeneity in colour, which indicates substantial variation in the distribution (i.e. concentration) of the cannabis extract throughout the plant material (see FIG. 1, panel A and B). Moreover, Product A has a visible oil-like residue absorbing through from the inside to the outside of the paper or wrap of the product, which indicates a substantial accumulation of cannabis extract to the point that it has saturated the paper (see FIG. 1, panel B).

As shown in FIG. 1 (panels A-B), extract-infused cannabis pre-rolled products produced using conventional techniques provide a product which does not exhibit homogeneous distribution of the cannabis extract, as evidenced by the distinct patches of dark coloured plant material, which a person of skill would appreciate is due to insufficient homogenization of the extract throughout the plant material. Furthermore, the pre-rolled joint of Product A contains a tightly packed cylinder of cannabis plant material. A person of skill in the art would appreciate that such a tightly packed form would impair consumption of the smokable product due to poor air circulation which in turn impairs burning (i.e. an uneven burn) when the product is consumed.

### Product B

Product B comprises an exemplary extract-infused pre-rolled joint of the present disclosure comprising brown paper, a red filter, and 35% total THC. The extract used for preparing the pre-rolled joint of Product B was a cannabis distillate. When the extract-infused cannabis plant material of Product B is exposed/removed, the rolled plant material does not retain any substantial shape and/or form (see FIG. 2, panel B). As can further be observed in FIG. 2B, the extract-infused cannabis plant material of Product B is substantially similar in colour. Indeed, the extract-infused cannabis plant material of Product B is substantially homogeneous in appearance. Moreover, Product B does not show any sign of an oil-like residue absorbing through from the inside to the outside of the paper or wrap of the product, rather Product B has a very clean, neat and uniform appearance and feel (see FIG. 2A).

As shown in FIG. 2 (panels A and B), extract-infused cannabis pre rolled products of the present disclosure exhibit homogeneous distribution of the cannabis extract throughout the cannabis plant materials. Furthermore, the pre-rolled joint of Product B contains loosely associated cannabis plant material, much like a pre-rolled joint containing natural flower or bud. A person of skill in the art would appreciate that a pre-rolled joint of Product B would burn evenly and provide a favorable consumption experience.

### Product C

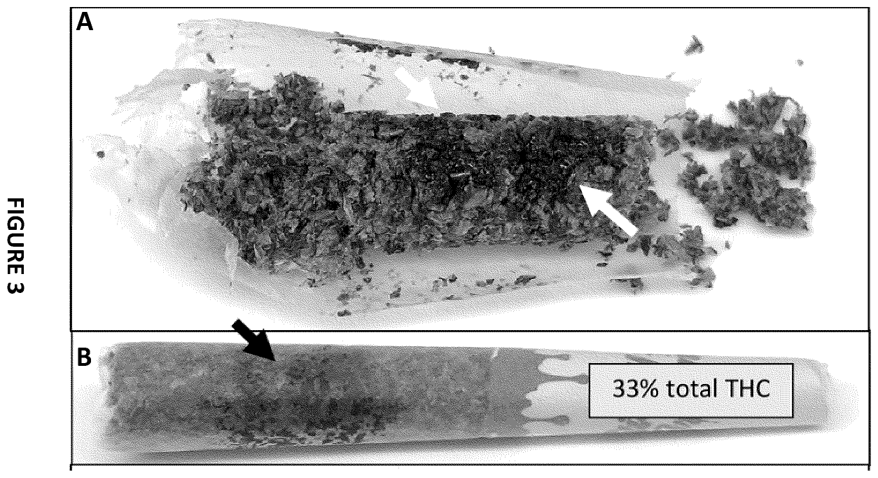

Product C is an extract-infused pre-rolled joint produced using a conventional method comprising white paper, a pink filter, and 33% total THC. The extract used for preparing the pre-rolled joint of Product C was a cannabis distillate. When the internal plant material of Product C is exposed/removed, the rolled plant material retains its cylinder shape (see FIG. 3, panel A). The extract-infused cannabis plant material of Product C exhibits heterogeneity in colour, which indicates substantial variation in the distribution (i.e. concentration) of the cannabis extract throughout the plant material (see FIG. 3, panels A and B). Moreover, Product C has a visible oil-like residue absorbing through from the inside to the outside of the paper or wrap of the product, which indicates a substantial accumulation of cannabis extract to the point that it has saturated the paper (see FIG. 3, panel B).

As shown in FIG. 1 (panels A-B), extract-infused cannabis pre-rolled products produced using conventional techniques provide a product which does not exhibit homogeneous distribution of the cannabis extract, as evidenced by the distinct patches of dark coloured plant material, which a person of skill would appreciate is due to insufficient homogenization of the extract throughout the plant material. Furthermore, the pre-rolled joint of Product A contains a tightly packed cylinder of cannabis plant material. A person of skill in the art would appreciate that such a tightly packed form would impair consumption of the smokable product due to poor air circulation which in turn impairs burning (i.e. an uneven burn) when the product is consumed.

### Product D

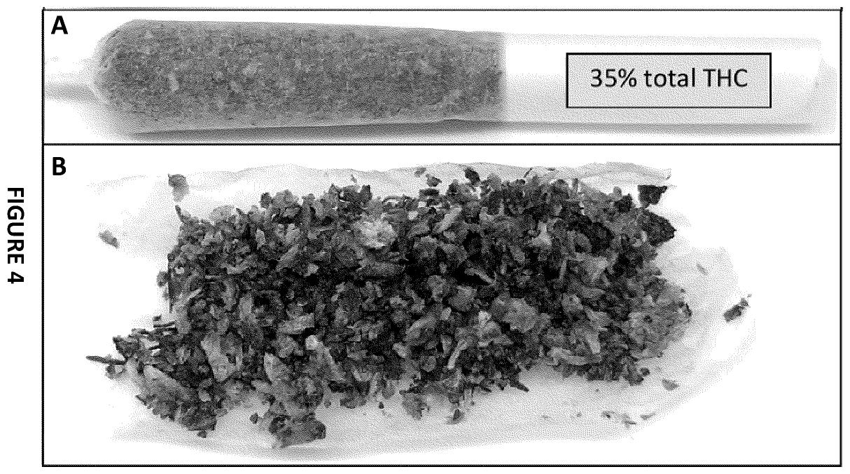

Product D comprises an exemplary extract-infused pre-rolled joint of the present disclosure comprising white paper, a white filter, and 35% total THC. The extract used for preparing the pre-rolled joint of Product D was a cannabis distillate. When the extract-infused cannabis plant material of Product D is exposed/removed, the rolled plant material exhibits only a slight retention of shape, owing predominantly to the more finely milled structure of the plant material for this cannabis cultivar (see FIG. 3). As can further be observed in FIG. 4B, the extract-infused cannabis biomass of Product D is substantially similar in colour. Indeed, the extract-infused cannabis biomass of Product D is substantially homogeneous in appearance. Moreover, Product D does not show any sign of an oil-like residue absorbing through from the inside to the outside of the paper or wrap of the product, rather Product D has a very clean, neat and uniform appearance and feel (see FIG. 4A).

As shown in FIG. 4 (panels A and B), extract-infused cannabis pre rolled products of the present disclosure exhibit homogeneous distribution of the cannabis extract throughout the cannabis plant materials. Furthermore, the pre-rolled joint of Product D contains loosely associated cannabis plant material, much like a pre-rolled joint containing natural flower or bud. A person of skill in the art would appreciate that a pre-rolled joint of Product D would burn evenly and provide a favorable consumption experience.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

As used herein, the term “substantially” refers to an approximately +/−5% variation from a given value. If a value is not used, then substantially means almost completely, but perhaps with some variation, contamination and/or additional component. In some embodiments, “substantially” may include completely.

It should be understood that the products and methods are described in terms of “comprising,” “containing,” or “including” various components or steps, the products and methods can also “consist essentially of” or “consist of the various components and steps”. Moreover, the indefinite articles “a” or “an”, as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b” or, “from a to b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be referenced herein, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method for preparing a homogeneous extract-infused cannabis biomass, the method comprising:
providing a cannabis extract having a predefined concentration of at least one extract-derived cannabinoid, at least one extract-derived terpene, or any combination thereof, wherein the cannabis extract is a distillate;
providing a cannabis plant material having a predefined concentration of at least one plant-derived cannabinoid, at least one plant-derived terpene, or any combination thereof, wherein the cannabis plant material is a milled or comminuted cannabis plant material;
in sequence:
i. heating the cannabis extract at a temperature of between about 60° C. and about 120° C. to provide a heated cannabis extract, wherein the heated cannabis extract is in a substantially liquid or flowable state;
ii. applying the heated cannabis extract, while in its heated state, to the cannabis plant material using a predefined ratio of the cannabis plant material to the heated cannabis extract to provide an extract-applied plant material, wherein the predefined ratio provides a target amount of at least one biomass cannabinoid, at least one biomass terpene, or any combination thereof within the extract-applied plant material, wherein the applying is by pouring, sprinkling or showering the heated cannabis extract onto the cannabis plant material with simultaneous mixing of the cannabis plant material; and
iii. continuing to mix the extract-applied plant material to provide a homogeneous extract-infused cannabis biomass, wherein there is less than a 15% variability in a target concentration of the at least one biomass cannabinoid, the at least one biomass terpene, or any combination thereof throughout a prepared batch and/or between alternate batches of the homogeneous extract-infused cannabis biomass.

2. (canceled)

3. The method of claim 1, wherein there is less than a 7.5% variability in the target concentration of the at least one biomass cannabinoid, the at least one biomass terpene, or any combination thereof throughout the prepared batch, between the alternate batches, or both.

4. The method of claim 1, wherein there is less than a 5% variability in the target concentration of the at least one biomass cannabinoid, the at least one biomass terpene, or any combination thereof throughout the prepared batch, between the alternate batches, or both.

5.-6. (canceled)

7. The method of claim 1, wherein there is between about 3% to about 7% variability in the target concentration of the at least one biomass cannabinoid, the at least one biomass terpene, or any combination thereof throughout the prepared batch, between the alternate batches, or both.

8.-10. (canceled)

11. The method of claim 1, wherein the target amount is of the at least one biomass cannabinoid, and the target amount is obtained by a combination of the at least one extract-derived cannabinoid and the at least one plant-derived cannabinoid.

12.-14. (canceled)

15. The method of claim 1, further comprising a step of providing at least one additive to the cannabis extract prior to the applying of the heated cannabis extract to the cannabis plant material.

16. The method of claim 15, wherein the at least one additive comprises at least one additive terpene, at least one additive cannabinoid, or any combination thereof, wherein the at least one additive is added to the cannabis extract at a predefined amount.

17.-25. (canceled)

26. The method of claim 1, wherein the step of mixing the extract-applied plant material comprises a step of freezing or cooling the extract-applied plant material.

27. The method of claim 26, which comprises freezing the extract-applied plant material at a temperature between about −30° C. to about −10° C. to produce a frozen extract-applied plant material.

28. The method of claim 27, further comprising mixing the frozen extract-applied plant material to provide the homogeneous extract-infused cannabis biomass.

29. The method of claim 1, further comprising, after the mixing, a step of screening the homogeneous extract-infused cannabis biomass to improve homogeneity.

30. The method of claim 1, wherein the applying is by pouring, sprinkling or showering the heated cannabis extract onto the cannabis plant material.

31. The method of claim 1, wherein the mixing is by rotational mixing, blending, stirring, beating, kneading, or any combination thereof of the extract-applied plant material, and is manual, machine operated and/or automated.

32. The method of claim 1, wherein the applying and the mixing steps are performed simultaneously.

33.-34. (canceled)

35. The method of claim 1, wherein in the step of heating the cannabis extract, the temperature is between about 60° C. and about 120° C.

36.-42. (canceled)

43. The method of claim 1, wherein the target amount of the biomass cannabinoid is of Δ9-THC, CBD, CBG, any neutral versions thereof, or any combination thereof.

44.-46. (canceled)

47. The method of claim 1, wherein the cannabis plant material is dried flower.

48. The method of claim 1, wherein the milled or comminuted cannabis plant material is of a particle size of less than or equal to 5.0 mm.

49. The method of claim 48, wherein the particle size is between about 100 μm and about 3.5 mm.

50. The method of claim 1, wherein a mass of the cannabis extract; the predefined concentration of the at least one extract-derived cannabinoid; the predefined concentration of the at least one extract-derived terpene; a mass of the cannabis plant material; the predefined concentration of the at least one plant-derived cannabinoid; the predefined concentration of the at least one plant-derived terpene; or any combination thereof is calculated using:
$$E = \frac{[ A \times B ] + [ C \times D ]}{[ A + C ]}$$
wherein A is the mass of the cannabis plant material, B is the concentration of the at least one plant-derived cannabinoid or the concentration of the at least one cannabis plant-derived terpene, C is the mass of the cannabis extract, D is the concentration of the at least one extract-derived cannabinoid or the concentration of the at least one extract-derived terpene, and E is the target concentration of the at least one biomass cannabinoid or the at least one biomass terpene.

51. The method of claim 1, wherein a length of time for the heating of the cannabis extract is calculated using:
$$The length of time for heating of the cannabis extract ( s ) = \frac{m \times cp \times Δ T}{P}$$
wherein m is a mass of the cannabis extract, cp is a specific heat capacity of the cannabis extract in J/kg° C., ΔT is a temperature change (° C.) of the cannabis extract; and P is a rate of energy (W) transfer to the cannabis extract.

52.-54. (canceled)

55. The method of claim 1, wherein the variability in the target concentration of the at least one biomass cannabinoid, the at least one biomass terpene, or any combination thereof is throughout the prepared batch and between alternate batches.

56.-89. (canceled)
